(19) United States
(12) Patent Application Publication
(10) Pub. No.: US 2026/0253738 A1
(43) Pub. Date: Aug. 27, 2026

(54) SYSTEM

(71) Applicant: SoftBank Group Corp., Tokyo (JP)

(72) Inventor: Hatsune OKUDAIRA, Tokyo (JP)

(73) Assignee: SoftBank Group Corp., Tokyo (JP)

(21) Appl. No.: 19/531,705

(22) Filed: Feb. 6, 2026

(30) Foreign Application Priority Data
Feb. 21, 2025 (JP) 2025-027008

(51) Int. Cl. G16H 50/30; G06Q 10/083; G06Q 50/12; G16H 10/65; G16H 20/60

(52) U.S. Cl. CPC G16H 50/30; G06Q 10/0843; G06Q 50/12; G16H 10/65; G16H 20/60

(57) ABSTRACT

The system according to the embodiment comprises an acquisition unit, an analysis unit, a provision unit, and a delivery unit. The acquisition unit acquires health information of a user. The analysis unit analyzes the user's preferences and nutritional balance based on the information acquired by the acquisition unit. The provision unit provides meal contents proposed by the analysis unit to a restaurant. The delivery unit delivers the meal provided by the provision unit to the user.

### CROSS-REFERENCE TO RELATED APPLICATION

The present application claims priority to and incorporates by reference the entire contents of Japanese Patent Application No. 2025-027008 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, services that propose appropriate meal contents and deliver them to users who have difficulty shopping have not been sufficiently provided, and there is room for improvement.

### SUMMARY OF THE INVENTION

The system according to the embodiment comprises an acquisition unit, an analysis unit, a provision unit, and a delivery unit. The acquisition unit acquires health information of a user. The analysis unit analyzes the user's preferences and nutritional balance based on the information acquired by the acquisition unit. The provision unit provides meal contents proposed by the analysis unit to a restaurant. The delivery unit delivers the meal provided by the provision unit to the user.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5 th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

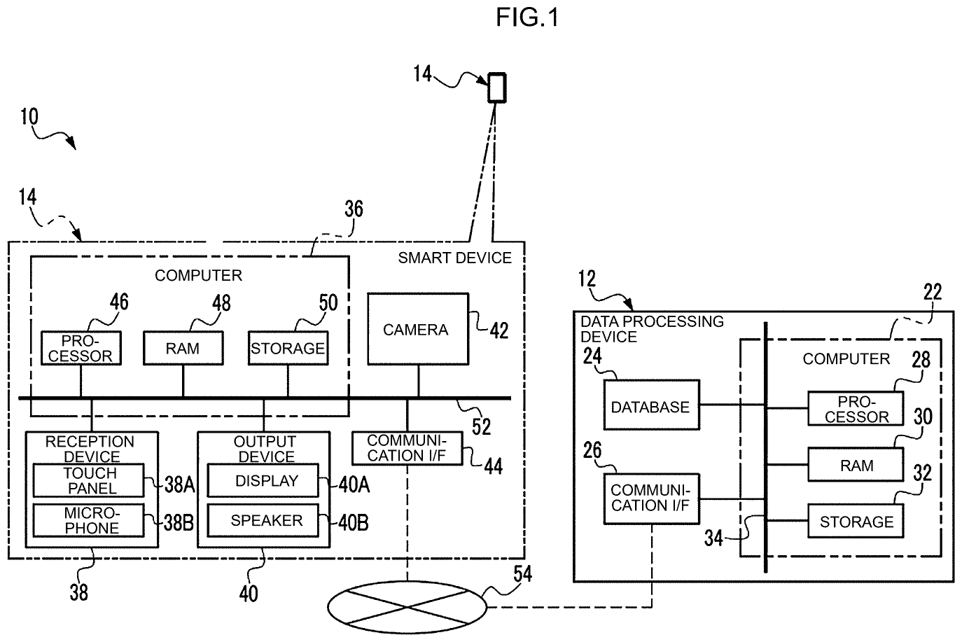

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

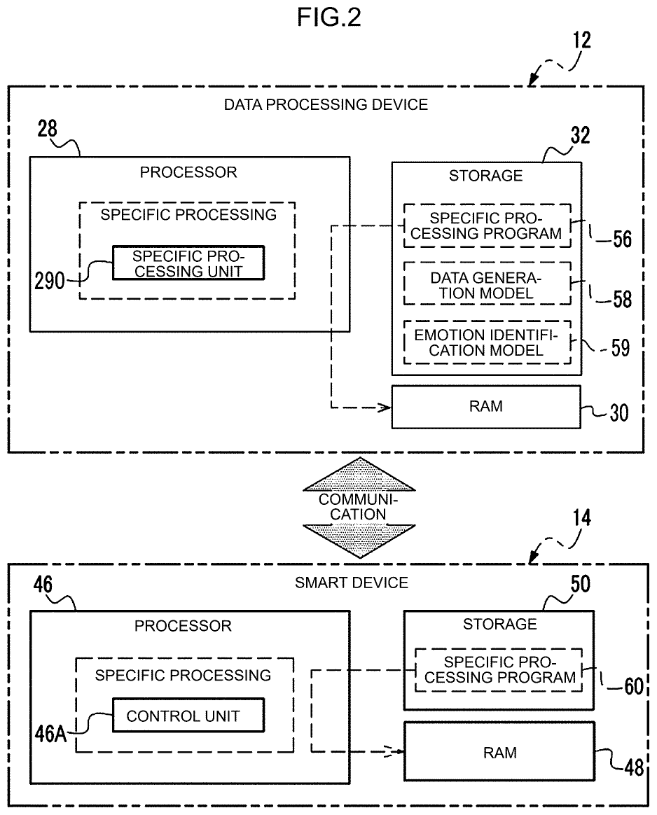

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is an interactive AI robot that proposes and delivers meals to elderly people who have difficulty shopping and residents of depopulated areas. This system allows a user to consult the AI robot by asking, for example, “What should I eat today?”, whereby the system proposes meal contents that take into account the user's preferences and nutritional balance, provides the proposed meal contents to a partner restaurant, and delivers the meal to the user's home. For example, when a user consults the AI robot by asking “What should I eat today?”, the user can communicate their preferences, allergy information, and health status to the AI robot. For instance, the user can express requests such as “I want to eat more vegetables today” or “I want to eat fish dishes.” Next, through dialogue with the user, the AI robot proposes meal contents that consider the user's preferences and nutritional balance. The AI robot generates optimal meal contents based on the user's past meal history and health status. For example, in response to a request for “I want to eat more vegetables,” the AI robot may propose menus such as salads or stir-fried vegetables. Furthermore, the system proposes meals that include the necessary nutrients, taking nutritional balance into account. The proposed meal contents are provided to partner restaurants. The restaurant prepares the meal based on the menu provided by the AI robot. For example, the restaurant cooks the proposed salad or stir-fried vegetables and delivers them to the user's home. As a result, even elderly people who have difficulty shopping or residents of depopulated areas can easily enjoy nutritionally balanced meals. Additionally, by proposing meals tailored to the user's preferences and health status, the system contributes to health maintenance. Thus, the interactive AI robot system can propose meal contents based on the user's health information, considering preferences and nutritional balance, provide them to restaurants, and deliver them to the user. Specifically, the system acquires natural language inquiries from the user (e.g., “What should I eat today?”) via a speech recognition module or text input interface, and generates input data such as a time-series array of character strings or audio spectral tensors (e.g., a string array of length 512 or audio waveform data with a sampling rate of 16 kHz) for input to a generative AI module. The system combines additional input features such as the user's preferences, allergy information, and health status (e.g., structured data vectors for weight, blood pressure, medical history, allergy ingredient list, etc.). The generative AI module, using, for example, a Transformer-based large language model or multimodal generative model, integrates the input user information and past meal history (e.g., a label array of meal contents for the past 30 days or a time-series vector of nutrient intake) as a multidimensional tensor, and outputs a list of meal proposal candidates (e.g., structured JSON data including dish name, estimated nutrient amount, allergy risk score, satisfaction prediction score for each candidate). Example outputs include “Salad (120 kcal, Vitamin C 30 mg, low allergy risk)” or “Grilled fish set meal (350 kcal, protein 25 g, medium allergy risk).” The system applies a scoring algorithm based on the user's health goals (e.g., salt reduction, low carbohydrate, specific nutrient enhancement) and preference patterns to these output candidates to select the optimal proposal. The selected meal proposal is automatically transmitted via API to the order management system of the partner restaurant, where the restaurant's cooking process management system automatically generates cooking instructions based on the received menu information. Furthermore, the system's delivery unit uses the user's address information, current location, and traffic data (e.g., map coordinate vectors, congestion scores from real-time traffic APIs) as input, and applies reinforcement learning algorithms or route optimization AI models (e.g., graph neural networks) to output the optimal delivery route (e.g., predicted time required for each delivery method, recommended route coordinate sequence). Example outputs include “Bike delivery: 15 minutes required, recommended route A” or “Drone delivery: 10 minutes required, recommended route B.” These AI outputs are used for subsequent processing via a threshold judgment module or user selection interface, and the final delivery method and estimated arrival time are determined. As a technical effect, the system not only automates simple menu recommendations and delivery arrangements by humans, but also achieves improvements in computer technology itself, such as increased proposal accuracy, efficiency in cooking and delivery processes, maximization of user satisfaction, and reduction of communication and computational load, by integrally analyzing multiple high-dimensional data (health information, preferences, history, geographic information, etc.) and performing optimization processing with AI models. Specific application fields include health maintenance support for homebound elderly, optimization of meal infrastructure in depopulated areas, automation of nutrition management in medical and nursing care settings, corporate welfare services, and meal supply support during disasters. Furthermore, by changing the architecture and learning methods of the AI model (e.g., transfer learning, multitask learning, data augmentation), flexible system expansion according to user segments and regional characteristics is possible.

The interactive AI robot system according to the embodiment comprises an acquisition unit, an analysis unit, a provision unit, and a delivery unit. The acquisition unit acquires health information of the user. The user's health information may include, for example, weight, blood pressure, meal contents, and amount of exercise, but is not limited thereto. The acquisition unit collects health information input by the user, for example. The acquisition unit can also acquire health information from wearable devices. For example, the acquisition unit collects weight and blood pressure data input by the user and provides them to the analysis unit. The acquisition unit also collects data such as amount of exercise and heart rate obtained from wearable devices and provides them to the analysis unit. The analysis unit analyzes the user's preferences and nutritional balance based on the information acquired by the acquisition unit. The analysis unit generates optimal meal contents based on the user's past meal history and health status, for example. The analysis unit uses generative AI to analyze the user's preferences and nutritional balance. For example, the analysis unit uses a generative AI model that takes the user's past meal history as input and outputs optimal meal contents to generate meal contents. The provision unit provides the meal contents proposed by the analysis unit to a restaurant. The provision unit automatically transmits the proposed meal contents to the restaurant, for example. The provision unit uses generative AI to provide the proposed meal contents to the restaurant. For example, the provision unit uses a generative AI model that takes the proposed meal contents as input and outputs a menu to be provided to the restaurant to generate the menu. The delivery unit delivers the meal provided by the provision unit to the user. The delivery unit delivers the meal prepared by the restaurant to the user's home, for example. The delivery unit uses generative AI to calculate the optimal delivery route. For example, the delivery unit uses a generative AI model that takes the user's address as input and outputs the optimal delivery route to calculate the delivery route. Thus, the interactive AI robot system according to the embodiment can propose meal contents based on the user's health information, considering preferences and nutritional balance, provide them to a restaurant, and deliver them to the user. Specifically, the interactive AI robot system collects health information input by the user (e.g., numerical vectors or categorical data for weight, blood pressure, meal contents, amount of exercise) in the acquisition unit, and integrates real-time data from wearable devices (e.g., time-series arrays of heart rate every minute, step count, estimated calorie consumption). The acquisition unit standardizes and normalizes these data, applies missing value imputation and outlier detection algorithms (e.g., Z-score method, IQR method), and generates high-quality input feature tensors. The analysis unit receives multidimensional health information tensors (e.g., three-dimensional tensors of [date, indicator type, value] for each user) and the user's past 30 days of meal history (e.g., dish ID array, nutrient intake vector) from the acquisition unit, and uses Transformer-based large language models or multimodal generative models to generate a list of meal proposal candidates that consider the user's preference patterns and health goals (e.g., salt reduction, low carbohydrate, specific nutrient enhancement). Within the AI model, feature extraction by self-attention mechanism, correlation analysis of history and health information by multi-head attention, and learning/inference using a composite objective function for proposal accuracy and nutritional balance (e.g., cross-entropy+nutrient deviation penalty) are performed. Example outputs include structured JSON data such as “Grilled fish set meal (350 kcal, protein 25 g, medium allergy risk, satisfaction prediction 0.92)” or “Vegetable salad (120 kcal, Vitamin C 30 mg, low allergy risk, satisfaction prediction 0.85).” The provision unit automatically transmits the meal proposal candidates received from the analysis unit to the order management API of partner restaurants, and the restaurant's cooking process management system automatically generates cooking instructions. Furthermore, the delivery unit uses the user's address information, current location, and traffic data (e.g., map coordinate vectors, congestion scores from real-time traffic APIs) as input, and applies graph neural networks and reinforcement learning algorithms to output the optimal delivery route (e.g., predicted time required for each delivery method, recommended route coordinate sequence). Example outputs include “Bike delivery: 15 minutes required, recommended route A” or “Drone delivery: 10 minutes required, recommended route B.” These AI outputs are used for subsequent processing via a threshold judgment module or user selection interface, and the final delivery method and estimated arrival time are determined. As a technical effect, the system not only automates simple menu recommendations and delivery arrangements by humans, but also achieves improvements in computer technology itself, such as increased proposal accuracy, efficiency in cooking and delivery processes, maximization of user satisfaction, and reduction of communication and computational load, by integrally analyzing multiple high-dimensional data (health information, preferences, history, geographic information, etc.) and performing optimization processing with AI models. Specific application fields include health maintenance support for homebound elderly, optimization of meal infrastructure in depopulated areas, automation of nutrition management in medical and nursing care settings, corporate welfare services, and meal supply support during disasters. Furthermore, by changing the architecture and learning methods of the AI model (e.g., transfer learning, multitask learning, data augmentation), flexible system expansion according to user segments and regional characteristics is possible.

The acquisition unit can acquire the user's preferences, allergy information, and health status. The acquisition unit collects preferences, allergy information, and health status input by the user, for example. For instance, the acquisition unit collects information on favorite and disliked ingredients and taste preferences input by the user. The acquisition unit can also collect allergy information input by the user. For example, the acquisition unit collects allergy information for specific ingredients input by the user. The acquisition unit can also collect health status input by the user. For example, the acquisition unit collects information on current medical history, presence of chronic diseases, and health checkup results input by the user. By acquiring the user's preferences, allergy information, and health status, the acquisition unit can propose more appropriate meal contents. Some or all of the above-described processing in the acquisition unit may be performed using AI or without using AI. For example, the acquisition unit can input preferences, allergy information, and health status provided by the user into generative AI and have the generative AI analyze the preferences, allergy information, and health status. Specifically, the acquisition unit accepts input data from the user such as text-based preference information (e.g., natural language sentences like “I like tomatoes” or “I don't like spicy food”), allergy information (e.g., keyword strings like “I am allergic to shrimp” or “I want to avoid wheat”), and health status (e.g., structured numerical vectors or categorical data such as weight 65 kg, blood pressure 120/80 mmHg, presence of diabetes). The acquisition unit preprocesses these input data by normalization and standardization in a preprocessing module, converts preference information into one-hot encoding or embedding vectors (e.g., ingredient ID vectors, taste feature vectors), organizes allergy information as binary flag arrays (e.g., 0/1 for each major allergen), and structures health status data as time-series tensors (e.g., arrays of weight, blood pressure, and blood glucose for the past 30 days) or category labels (e.g., presence of medical history). The acquisition unit integrates these diverse data and generates input tensors for the AI model (e.g., multidimensional arrays concatenating preference features, allergy flags, and health status vectors for each user). The AI model, such as a Transformer-based large language model or multimodal encoder, processes the user's natural language input and structured health data simultaneously. Example inputs include “Favorite ingredient: tomato, disliked ingredient: green pepper, allergy: shrimp, health status: diabetes, weight 65 kg.” The AI model extracts the user's preference patterns and health risks from these inputs and outputs structured data such as preference cluster labels (e.g., Japanese food preference, low-carb orientation), allergy risk scores (e.g., risk values for each major allergen), and health status summaries (e.g., recommended level of nutritional restriction, disease risk prediction values). Example outputs include “Preference cluster: Japanese food, allergy risk: high for shrimp, low for wheat, health recommendation: recommend low-carb menu.” These outputs are input to subsequent meal proposal AI and nutritional balance optimization modules and used to generate optimized meal contents for each user. As a technical effect, the acquisition unit, unlike simple human questionnaire aggregation, achieves essential improvements in computer technology such as increased proposal accuracy, maximized personalization for each user, reduced data input burden, and early detection of health risks by integrating and analyzing multidimensional and diverse health-related data with high precision and in real time. Specific application fields include health maintenance support for homebound elderly, nutrition management for patients requiring dietary restrictions, corporate welfare services, allergy management for school lunches, and performance optimization for athletes. Furthermore, by changing the architecture and feature design of the AI model, flexible system expansion according to user segments, regional characteristics, and disease types is possible.

The analysis unit can generate appropriate meal contents based on the user's past meal history and health status. The analysis unit generates optimal meal contents based on the user's past meal history and health status, for example. For instance, the analysis unit collects and analyzes the user's past meal history. For example, the analysis unit collects and analyzes the contents, frequency, and timing of meals the user has eaten in the past. The analysis unit can also collect and analyze the user's health status. For example, the analysis unit collects and analyzes the user's current medical history, presence of chronic diseases, and health checkup results. By generating optimal meal contents based on the user's past meal history and health status, the analysis unit can propose meals tailored to the user. Some or all of the above-described processing in the analysis unit may be performed using AI or without using AI. For example, the analysis unit can input the user's past meal history and health status into generative AI and have the generative AI generate optimal meal contents. Specifically, the analysis unit structures time-series meal history data for each user (e.g., array of meal content IDs for the past 30 days, nutrient intake vectors, meal time timestamp arrays) and health status data (e.g., time-series vectors for weight, blood pressure, blood glucose, category labels for medical history, numerical vectors for health checkup results) as input tensors for the AI model. The analysis unit inputs these multidimensional data into Transformer-based large language models or multimodal generative models, and extracts correlation patterns between meal history and health status in high-dimensional space using self-attention mechanisms and multi-head attention. Example inputs include “Past 30 days of meals: grilled fish set meal 10 times, vegetable salad 5 times, curry rice 3 times, average calorie intake 1800 kcal, weight 65 kg, blood pressure 120/80 mmHg, diabetes present.” The AI model extracts preference tendencies (e.g., Japanese food orientation, insufficient vegetable intake) and health risks (e.g., high blood sugar risk, excessive salt intake risk) from these inputs and outputs a list of meal proposal candidates (e.g., structured JSON data including dish name, estimated nutrient amount, allergy risk score, satisfaction prediction score). Example outputs include “Grilled fish set meal (350 kcal, protein 25 g, medium allergy risk, satisfaction prediction 0.92)” and “Vegetable salad (120 kcal, Vitamin C 30 mg, low allergy risk, satisfaction prediction 0.85).” The analysis unit applies scoring algorithms based on health goals (e.g., salt reduction, low carbohydrate, specific nutrient enhancement) and preference patterns to these outputs to select the optimal proposal. Within the AI model, learning and inference are performed using a composite objective function for proposal accuracy and nutritional balance (e.g., cross-entropy+nutrient deviation penalty) as the loss function. These AI outputs are input to subsequent provision units and user interfaces and used to present optimized meal proposals for each user and generate order data for restaurants. As a technical effect, the analysis unit, unlike simple human history aggregation or rule-based proposals, achieves essential improvements in computer technology such as increased proposal accuracy, reduced health risks, maximized user satisfaction, and improved computational efficiency by integrally analyzing vast time-series data and health indicators in high-dimensional space and performing optimization processing with AI models. Specific application fields include health maintenance support for homebound elderly, meal management for patients with lifestyle diseases, nutrition optimization for athletes, corporate welfare services, and nutrition management for school lunches. Furthermore, by changing the architecture and learning methods of the AI model (e.g., transfer learning, multitask learning, data augmentation), flexible system expansion according to user segments, disease types, and regional characteristics is possible.

The provision unit can automatically transmit the proposed meal contents to the restaurant. The provision unit automatically transmits the proposed meal contents to the restaurant, for example. For instance, the provision unit uses a generative AI model that takes the proposed meal contents as input and outputs a menu to be provided to the restaurant to generate the menu. The provision unit may transmit meal contents when specific conditions are met. The provision unit can also transmit meal contents periodically. For example, the provision unit transmits meal contents at a fixed time every day. By automatically transmitting the proposed meal contents to the restaurant, the provision unit enables rapid meal preparation. Some or all of the above-described processing in the provision unit may be performed using AI or without using AI. For example, the provision unit can input the proposed meal contents into generative AI and have the generative AI generate the menu to be provided to the restaurant. Specifically, the provision unit takes the list of meal proposal candidates received from the analysis unit (e.g., structured JSON data including dish name, estimated nutrient amount, allergy risk score, satisfaction prediction score) as input and automatically converts it into a format compatible with the restaurant's order management API. The provision unit adds detailed information such as required ingredient lists, cooking procedures, allergy precautions, and recommended serving times for each meal content, enabling the restaurant's cooking process management system to automatically generate cooking instructions. The AI model may use a large language model for menu generation or a rule-based template engine to output optimal menu instruction data that considers operational requirements for each restaurant (e.g., cooking equipment constraints, inventory status, business hours) from the input meal proposal data. Example inputs include “Grilled fish set meal (350 kcal, medium allergy risk, cooking time 15 minutes)” and “Vegetable salad (120 kcal, low allergy risk, cooking time 5 minutes).” The AI model generates optimal cooking instructions for each restaurant from these inputs (e.g., ingredient order list, cooking procedure, allergy warning label, recommended serving time), and example outputs include “Store A: grilled fish set meal, cooking instruction: grill fish for 15 minutes, allergy warning: no shrimp, serving time 12:00” and “Store B: vegetable salad, cooking instruction: cut vegetables in 5 minutes, allergy warning: no wheat, serving time 18:00.” These outputs are automatically linked to the restaurant's cooking process management system and inventory management system, realizing automation and efficiency in cooking and serving processes. Furthermore, the provision unit can incorporate periodic scheduling functions and conditional branching logic (e.g., prioritize special menus when the user's health status deteriorates). As a technical effect, the provision unit, unlike simple human order transmission or manual operations, achieves essential improvements in computer technology such as reduction of order errors, efficiency in cooking and serving processes, increased user satisfaction, and reduction of communication and computational load by realizing optimization processing under various constraint conditions, real-time data linkage, and automation of cooking processes using AI models. Specific application fields include meal delivery for homebound elderly, corporate welfare services, automatic ordering for school lunches, meal supply support during disasters, and automatic operation optimization for restaurant chains. Furthermore, by changing the AI model's algorithm or API specifications, flexible system expansion according to restaurant scale, regional characteristics, and user segments is possible.

The delivery unit can deliver the meal prepared by the restaurant to the user's home. The delivery unit delivers the meal prepared by the restaurant to the user's home, for example. For instance, the delivery unit uses a generative AI model that takes the meal prepared by the restaurant as input and outputs the optimal delivery route to calculate the delivery route. The delivery unit may use delivery means such as drones, bikes, or cars to deliver the meal. The delivery unit can also adjust the delivery time. For example, the delivery unit delivers the meal at the time specified by the user. By delivering the meal prepared by the restaurant to the user's home, the delivery unit enables the user to easily receive the meal. Some or all of the above-described processing in the delivery unit may be performed using AI or without using AI. For example, the delivery unit can input the meal prepared by the restaurant into generative AI and have the generative AI calculate the optimal delivery route. Specifically, the delivery unit structures input tensors for the AI model by integrating meal information received from the restaurant (e.g., menu ID, cooking completion time, allergy precautions), user address information (e.g., map coordinate vectors, building type, floor number), current location information, traffic data (e.g., congestion scores from real-time traffic APIs, weather information), and delivery means operation status (e.g., drone battery level, current location of bikes, number of vehicles). The delivery unit inputs these multidimensional data into a delivery route optimization AI model using graph neural networks or reinforcement learning algorithms, and outputs predicted time required for each delivery means, recommended route coordinate sequence, and predicted delivery cost. Example inputs include “Delivery destination: 1-1-1 Chiyoda-ku, Tokyo, current location: Store A, traffic congestion: 0.8, weather: rain, drone battery: 80%, number of bikes: 2.” The AI model generates outputs such as “Bike delivery: 15 minutes required, recommended route A” and “Drone delivery: 10 minutes required, recommended route B” from these inputs, and uses them to select the delivery means and determine the delivery route. Furthermore, the delivery unit can apply delivery scheduling algorithms that consider the user's specified time and desired receipt conditions (e.g., delivery left at the front door, contactless receipt). The AI output is used for subsequent processing via a threshold judgment module or user selection interface, and the final delivery means and estimated arrival time are determined. As a technical effect, the delivery unit, unlike simple human delivery arrangements or rule-based route selection, achieves essential improvements in computer technology such as shortened delivery time, cost reduction, improved delivery efficiency, maximized user satisfaction, and reduction of communication and computational load by integrally analyzing vast geographic, traffic, and delivery means status information in high-dimensional space and performing optimization processing with AI models. Specific application fields include meal delivery for homebound elderly, last-mile delivery in depopulated areas, corporate welfare services, emergency meal supply during disasters, and automatic delivery optimization for restaurant chains. Furthermore, by changing the AI model's algorithm or delivery means management module, flexible system expansion according to regional characteristics, user segments, and delivery infrastructure is possible.

The acquisition unit can estimate the user's emotion and adjust the timing of acquiring health information based on the estimated emotion of the user. The acquisition unit estimates the user's emotion and adjusts the timing of acquiring health information based on the estimated emotion, for example. For instance, if the user is feeling stressed, the acquisition unit delays the acquisition timing to obtain health information in a relaxed state. If the user is relaxed, the acquisition unit can acquire health information immediately and start analysis promptly. Furthermore, if the user is in a hurry, the acquisition unit can acquire health information in a short time and perform rapid analysis. By adjusting the timing of acquiring health information based on the user's emotion, the acquisition unit can acquire health information at a more appropriate timing. Emotion estimation is realized using, for example, an emotion engine or generative AI with emotion estimation functions. Generative AI may be a text generative AI (e.g., LLM) or a multimodal generative AI, but is not limited thereto. Some or all of the above-described processing in the acquisition unit may be performed using AI or without using AI. For example, the acquisition unit can input the user's emotion data into generative AI and have the generative AI estimate the emotion. Specifically, the acquisition unit accepts input data from the user such as audio data (e.g., speech audio spectral tensor, 3 seconds in length, 16 kHz sampling rate), facial expression images (e.g., face image 128×128 pixels RGB tensor), and text chat history (e.g., natural language sentences such as “I'm tired today” or “I'm feeling down”). The acquisition unit preprocesses these multimodal data in a preprocessing module by noise removal and normalization, extracts audio features (e.g., MFCC vectorization), image features (e.g., facial landmark detection), and text embeddings (e.g., BERT-based contextual vectorization). The acquisition unit concatenates these features into a multidimensional tensor (e.g., audio 128 dimensions+image 256 dimensions+text 768 dimensions=total 1152 dimensions) and inputs it into an emotion estimation AI model. The AI model may use a multimodal Transformer or a combination of convolutional neural networks and recurrent neural networks to output emotion categories (e.g., stress, relaxation, urgency, joy, sadness) and emotion intensity scores (e.g., stress level 0.85, relaxation level 0.10) from the input data. Example outputs include “Emotion label: stress, intensity 0.78” and “Emotion label: relaxation, intensity 0.92.” The acquisition unit inputs these AI outputs into a threshold judgment module and applies rule-based control such as delaying health information acquisition by 10 minutes if the stress level is high, acquiring immediately if the relaxation level is high, and shortening the acquisition interval if the urgency level is high. Furthermore, the acquisition unit can implement adaptive scheduling by referring to the user's past emotion transition history (e.g., time-series vector of emotion scores for the past 7 days) and lowering the acquisition frequency if emotion fluctuations are large. As a technical effect, the acquisition unit, unlike simple human timer control or uniform acquisition, achieves essential improvements in computer technology such as reduced user burden, improved reliability of acquired data, suppression of unnecessary data collection, and optimization of system-wide computational and communication load by combining multidimensional emotion analysis with dynamic scheduling using AI models. Specific application fields include health monitoring for homebound elderly, workplace environments where stress management is important, condition management for athletes, patient status monitoring in telemedicine, and mental health support systems. Furthermore, by changing the architecture and feature design of the emotion estimation AI model (e.g., weighting adjustment for audio, image, and text), and scheduling algorithms (e.g., optimization by reinforcement learning), flexible system expansion according to user segments and usage environments is possible.

The acquisition unit can analyze the user's past health information and select an appropriate acquisition method. The acquisition unit analyzes the user's past health information and selects the optimal acquisition method, for example. For instance, the acquisition unit proposes the optimal method based on the user's past health information acquisition methods (manual input, wearable devices, etc.). The acquisition unit can also select a method to acquire health information at specific times based on the user's past health information. Furthermore, the acquisition unit can analyze the user's past health information and select the most efficient acquisition method. By analyzing the user's past health information, the acquisition unit can select the optimal acquisition method. Some or all of the above-described processing in the acquisition unit may be performed using AI or without using AI. For example, the acquisition unit can input the user's past health information into generative AI and have the generative AI select the optimal acquisition method. Specifically, the acquisition unit collects health information acquisition history data for each user over the past year (e.g., acquisition method ID, acquisition date and time, acquisition success rate, data accuracy score, user response time, etc., in a structured table). The acquisition unit inputs these history data as time-series vectors or category label arrays (e.g., manual input 10 times, wearable device 50 times, smartphone app 20 times) into the AI model. The AI model may use a recurrent neural network for time-series analysis or a decision tree-based gradient boosting model to output effectiveness scores for each acquisition method (e.g., accuracy 0.95, response rate 0.80, user burden 0.20) and optimal acquisition timing (e.g., 7:00 AM, 9:00 PM) from the input data. Example inputs include “Past 30 days: wearable device acquisition success rate 98%, manual input success rate 70%, high data accuracy when acquired in the morning.” The AI model generates outputs such as “Recommended acquisition method: wearable device, recommended acquisition time: 7:00 AM, predicted accuracy 0.97” from these inputs. The acquisition unit automatically selects the optimal acquisition method and timing for each user based on the AI output and dynamically changes system settings. Furthermore, if the acquisition unit detects changes in the user's lifestyle (e.g., introduction of new devices, changes in daily rhythm), it can perform retraining or reevaluation and continuously optimize the acquisition method. As a technical effect, the acquisition unit, unlike human heuristics or uniform settings, achieves essential improvements in computer technology such as improved data acquisition accuracy, reduced user burden, minimized acquisition failure rate, and reduced system operation costs by analyzing history data and performing dynamic optimization with AI models. Specific application fields include home health management systems, corporate health management support, patient monitoring in medical institutions, training management for athletes, and data acquisition optimization in telemedicine. Furthermore, by changing the AI model's algorithm or feature design (e.g., addition of user attributes, device performance, environmental factors), and acquisition method selection logic (e.g., dynamic optimization by multi-armed bandit), flexible system expansion according to user segments and usage environments is possible.

The acquisition unit can perform filtering based on the user's current lifestyle and meal preferences when acquiring health information. The acquisition unit performs filtering based on the user's current lifestyle and meal preferences when acquiring health information, for example. For instance, the acquisition unit acquires appropriate health information based on the user's current lifestyle (work, vacation, etc.). The acquisition unit can also preferentially acquire relevant health information based on the user's meal preferences. Furthermore, the acquisition unit can filter out unnecessary information based on the user's current lifestyle and meal preferences. By performing filtering based on the user's current lifestyle and meal preferences, the acquisition unit can eliminate unnecessary information. Some or all of the above-described processing in the acquisition unit may be performed using AI or without using AI. For example, the acquisition unit can input the user's current lifestyle and meal preferences into generative AI and have the generative AI perform filtering. Specifically, the acquisition unit accepts input data from the user such as lifestyle (e.g., occupation category, working hours, holiday information, travel flag as category data), meal preferences (e.g., Japanese food orientation, vegetarian, preference for spicy food as labels), and current activity status (e.g., exercising, resting, going out as state labels). The acquisition unit encodes these information as one-hot encoding or embedding vectors, combines them with a list of candidate health information to be acquired (e.g., array of items such as weight, blood pressure, blood glucose, meal contents, amount of exercise), and inputs them into the AI model. The AI model may use a combination of rule-based filtering and large language models or a multimodal encoder to output “acquisition priority score” and “acquisition unnecessary flag” from the input data. Example inputs include “Lifestyle: working, preference: Japanese food, activity: going out.” The AI model generates outputs such as “Priority acquisition: blood pressure, step count; unnecessary acquisition: meal contents” from these inputs. The acquisition unit suppresses unnecessary health information acquisition requests and efficiently collects only necessary information based on the AI output. Furthermore, the acquisition unit can learn from the user's past filtering history and acquisition results (e.g., acquisition success rate, user satisfaction score) to continuously improve filtering accuracy. As a technical effect, the acquisition unit, unlike simple human item selection or uniform acquisition, achieves essential improvements in computer technology such as reduced data noise, minimized user burden, optimized system-wide computational and communication load, and improved usefulness of acquired data by performing multidimensional data analysis and dynamic filtering with AI models. Specific application fields include home health management, corporate health management support, performance management for athletes, data acquisition optimization in telemedicine, and personalized nutrition guidance. Furthermore, by changing the AI model's algorithm or feature design (e.g., addition of lifestyle, preferences, environmental factors), and filtering logic (e.g., optimization by reinforcement learning), flexible system expansion according to user segments and usage environments is possible.

The acquisition unit can estimate the user's emotion and determine the priority of health information to be acquired based on the estimated emotion of the user. The acquisition unit estimates the user's emotion and determines the priority of health information to be acquired based on the estimated emotion, for example. For instance, if the user is feeling stressed, the acquisition unit preferentially acquires stress-related health information. If the user is relaxed, the acquisition unit can preferentially acquire general health information. Furthermore, if the user is in a hurry, the acquisition unit can preferentially acquire important health information. By determining the priority of health information to be acquired based on the user's emotion, the acquisition unit can preferentially acquire important information. Emotion estimation is realized using, for example, an emotion engine or generative AI with emotion estimation functions. Generative AI may be a text generative AI (e.g., LLM) or a multimodal generative AI, but is not limited thereto. Some or all of the above-described processing in the acquisition unit may be performed using AI or without using AI. For example, the acquisition unit can input the user's emotion data into generative AI and have the generative AI estimate the emotion. Specifically, the acquisition unit accepts input data from the user such as audio data (e.g., speech audio spectral tensor, 3 seconds in length, 16 kHz sampling rate), facial expression images (e.g., face image 128×128 pixels RGB tensor), and text chat history (e.g., natural language sentences such as “I'm tired today” or “I'm feeling down”). The acquisition unit preprocesses these multimodal data in a preprocessing module by noise removal and normalization, extracts audio features (e.g., MFCC vectorization), image features (e.g., facial landmark detection), and text embeddings (e.g., BERT-based contextual vectorization). The acquisition unit concatenates these features into a multidimensional tensor (e.g., audio 128 dimensions+image 256 dimensions+text 768 dimensions=total 1152 dimensions) and inputs it into an emotion estimation AI model. The AI model may use a multimodal Transformer or a combination of convolutional neural networks and recurrent neural networks to output emotion categories (e.g., stress, relaxation, urgency, joy, sadness) and emotion intensity scores (e.g., stress level 0.85, relaxation level 0.10) from the input data. Example outputs include “Emotion label: stress, intensity 0.78” and “Emotion label: relaxation, intensity 0.92.” The acquisition unit inputs these AI outputs into a threshold judgment module and dynamically determines the priority of health information acquisition requests, such as preferentially acquiring stress-related health information (e.g., heart rate, blood pressure, sleep data) if the stress level is high, general health information (e.g., weight, meal contents) if the relaxation level is high, and important health information (e.g., blood glucose, allergy reactions) if the urgency level is high. Furthermore, the acquisition unit can implement adaptive priority control by linking the user's past emotion transition history (e.g., time-series vector of emotion scores for the past 7 days) and health information acquisition history, such as increasing the acquisition frequency of stress-related items if emotion fluctuations are large. As a technical effect, the acquisition unit, unlike simple human timer control or uniform acquisition, achieves essential improvements in computer technology such as reduced user burden, improved reliability of acquired data, prevention of missing important information, and optimization of system-wide computational and communication load by combining multidimensional emotion analysis with dynamic priority control using AI models. Specific application fields include health monitoring for homebound elderly, workplace environments where stress management is important, condition management for athletes, patient status monitoring in telemedicine, and mental health support systems. Furthermore, by changing the architecture and feature design of the emotion estimation AI model (e.g., weighting adjustment for audio, image, and text), and priority determination algorithms (e.g., optimization by reinforcement learning), flexible system expansion according to user segments and usage environments is possible.

The acquisition unit can preferentially acquire highly relevant information by considering the user's geographic location information when acquiring health information. The acquisition unit preferentially acquires highly relevant information by considering the user's geographic location information when acquiring health information, for example. For instance, if the user is in a specific region, the acquisition unit preferentially acquires health information related to that region. The acquisition unit can also acquire optimal health information based on the user's geographic location information. Furthermore, the acquisition unit can filter out unnecessary information by considering the user's geographic location information. By considering the user's geographic location information, the acquisition unit can preferentially acquire highly relevant information. Some or all of the above-described processing in the acquisition unit may be performed using AI or without using AI. For example, the acquisition unit can input the user's geographic location information into generative AI and have the generative AI acquire highly relevant information. Specifically, the acquisition unit accepts input data from the user such as geographic location information (e.g., GPS coordinate vector, address string, region ID), current environmental information (e.g., environmental sensor data such as temperature, humidity, air pollution level, pollen count), and region-specific health risk information (e.g., public data such as influenza prevalence, heatstroke alert level). The acquisition unit preprocesses these information in a preprocessing module by normalization and standardization, and generates geographic feature vectors (e.g., latitude, longitude, altitude, region cluster ID). The acquisition unit combines these with a list of candidate health information to be acquired (e.g., array of items such as body temperature, blood pressure, allergy symptoms, respiratory rate) and inputs them into the AI model. The AI model may use a large language model with a geographic information embedding layer or a multimodal encoder to output “acquisition priority score” and “acquisition unnecessary flag” from the input data. Example inputs include “Current location: Shinjuku-ku, Tokyo, temperature 35° C., high pollen count, medium influenza prevalence.” The AI model generates outputs such as “Priority acquisition: body temperature, respiratory rate, allergy symptoms; unnecessary acquisition: blood glucose” from these inputs. The acquisition unit suppresses unnecessary health information acquisition requests and preferentially collects information related to region-specific health risks based on the AI output. Furthermore, the acquisition unit can learn from the user's past geographic movement history and acquisition results (e.g., acquisition success rate, user satisfaction score) to continuously improve filtering accuracy and priority determination accuracy. As a technical effect, the acquisition unit, unlike simple human item selection or uniform acquisition, achieves essential improvements in computer technology such as reduced data noise, minimized user burden, optimized system-wide computational and communication load, and improved usefulness of acquired data by performing multidimensional geographic information analysis and dynamic filtering with AI models. Specific application fields include home health management, corporate health management support, performance management for athletes, data acquisition optimization in telemedicine, and region-specific health risk management. Furthermore, by changing the AI model's algorithm or feature design (e.g., addition of geographic and environmental factors), and filtering logic (e.g., optimization by reinforcement learning), flexible system expansion according to user segments and usage environments is possible.

The acquisition unit can analyze the user's social media activity and acquire relevant information when acquiring health information. The acquisition unit analyzes the user's social media activity and acquires relevant information when acquiring health information, for example. For instance, the acquisition unit analyzes health-related posts from the user's social media activity and acquires relevant information. The acquisition unit can also acquire health information based on the user's social media activity. Furthermore, the acquisition unit can analyze the user's social media activity and filter out unnecessary information. By analyzing the user's social media activity, the acquisition unit can acquire relevant health information. Some or all of the above-described processing in the acquisition unit may be performed using AI or without using AI. For example, the acquisition unit can input the user's social media activity into generative AI and have the generative AI acquire relevant information. Specifically, the acquisition unit accepts input data from the user such as social media post data (e.g., text posts, image posts, video posts, post time, hashtags, number of likes, number of comments as structured data), follow relationships and trend information (e.g., restaurant IDs followed, health-related influencer IDs, trending health topic labels). The acquisition unit preprocesses these data using natural language processing modules and image analysis modules to generate text embedding vectors (e.g., BERT-based contextual vectors), image feature vectors (e.g., ResNet-based image embeddings), and post metadata vectors (e.g., post frequency, time distribution). The acquisition unit concatenates these features into a multidimensional tensor (e.g., text 768 dimensions+image 256 dimensions+metadata 32 dimensions=total 1056 dimensions) and inputs it into the AI model. The AI model may use a multimodal Transformer or graph neural network to output extraction of health-related posts, health status estimation, acquisition priority score, and acquisition unnecessary flag from the input data. Example inputs include “Recent posts: ‘I've been feeling unwell lately’, ‘On a diet’, image: salad photo, hashtag: #healthconscious.” The AI model generates outputs such as “Priority acquisition: weight, meal contents, amount of exercise; unnecessary acquisition: blood pressure” from these inputs. The acquisition unit suppresses unnecessary health information acquisition requests and preferentially collects information related to the user's interests and health status based on the AI output. Furthermore, the acquisition unit can learn from the user's past social media activity history and acquisition results (e.g., acquisition success rate, user satisfaction score) to continuously improve filtering accuracy and priority determination accuracy. As a technical effect, the acquisition unit, unlike simple human item selection or uniform acquisition, achieves essential improvements in computer technology such as reduced data noise, minimized user burden, optimized system-wide computational and communication load, and improved usefulness of acquired data by performing multidimensional social data analysis and dynamic filtering with AI models. Specific application fields include home health management, corporate health management support, personalized nutrition guidance, data acquisition optimization in telemedicine, and health trend analysis. Furthermore, by changing the AI model's algorithm or feature design (e.g., addition of post content, images, trend factors), and filtering logic (e.g., optimization by reinforcement learning), flexible system expansion according to user segments and usage environments is possible.

The analysis unit can estimate the user's emotion and adjust the expression method of analysis based on the estimated emotion of the user. The analysis unit estimates the user's emotion and adjusts the expression method of analysis based on the estimated emotion, for example. For instance, if the user is relaxed, the analysis unit provides detailed analysis results. If the user is in a hurry, the analysis unit can provide concise analysis results. Furthermore, if the user is feeling stressed, the analysis unit can provide visually easy-to-understand analysis results. By adjusting the expression method of analysis based on the user's emotion, the analysis unit can provide more appropriate analysis results. Emotion estimation is realized using, for example, an emotion engine or generative AI with emotion estimation functions. Generative AI may be a text generative AI (e.g., LLM) or a multimodal generative AI, but is not limited thereto. Some or all of the above-described processing in the analysis unit may be performed using AI or without using AI. For example, the analysis unit can input the user's emotion data into generative AI and have the generative AI estimate the emotion. Specifically, the analysis unit accepts input data from the user such as audio data (e.g., speech audio spectral tensor, 3 seconds in length, 16 kHz sampling rate), facial expression images (e.g., face image 128×128 pixels RGB tensor), and text chat history (e.g., natural language sentences such as “I'm tired today” or “I'm feeling down”). The analysis unit preprocesses these multimodal data in a preprocessing module by noise removal and normalization, extracts audio features (e.g., MFCC vectorization), image features (e.g., facial landmark detection), and text embeddings (e.g., BERT-based contextual vectorization). The analysis unit concatenates these features into a multidimensional tensor (e.g., audio 128 dimensions+image 256 dimensions+text 768 dimensions=total 1152 dimensions) and inputs it into an emotion estimation AI model. The AI model may use a multimodal Transformer or a combination of convolutional neural networks and recurrent neural networks to output emotion categories (e.g., stress, relaxation, urgency, joy, sadness) and emotion intensity scores (e.g., stress level 0.85, relaxation level 0.10) from the input data. Example outputs include “Emotion label: stress, intensity 0.78” and “Emotion label: relaxation, intensity 0.92.” The analysis unit inputs these AI outputs into a threshold judgment module and dynamically switches the expression method, such as generating visually easy-to-understand analysis results using graphs or icons if the stress level is high, generating analysis results with detailed numerical values and explanatory text if the relaxation level is high, and presenting only key points in bullet points if the urgency level is high. Furthermore, the analysis unit can refer to the user's past emotion transition history and analysis result viewing history (e.g., time-series vector of emotion scores for the past 7 days, distribution of analysis result viewing times) to learn and adapt the optimal expression style for each user. As a technical effect, the analysis unit, unlike simple human template switching or uniform output, achieves essential improvements in computer technology such as reduced user burden, improved understanding of analysis results, maximized information transmission efficiency, and optimized system-wide computational and communication load by combining multidimensional emotion analysis with dynamic expression optimization using AI models. Specific application fields include home health management systems, analysis reports for patients in telemedicine, corporate health management support, performance analysis for athletes, and mental health support systems. Furthermore, by changing the architecture of the emotion estimation AI model and expression optimization algorithms (e.g., user response optimization by reinforcement learning), flexible system expansion according to user segments and usage environments is possible.

The analysis unit can adjust the level of detail of analysis based on the importance of health information during analysis. The analysis unit adjusts the level of detail of analysis based on the importance of health information during analysis, for example. For instance, the analysis unit performs detailed analysis for important health information. The analysis unit can perform concise analysis for general health information. Furthermore, the analysis unit can adjust the level of detail of analysis based on the importance of health information. By adjusting the level of detail of analysis based on the importance of health information, the analysis unit can analyze important information in detail. Some or all of the above-described processing in the analysis unit may be performed using AI or without using AI. For example, the analysis unit can input the importance of health information into generative AI and have the generative AI adjust the level of detail of analysis. Specifically, the analysis unit accepts input data from the user such as importance scores for each health information item (e.g., priority labels set by doctors or registered dietitians, automatic scoring values by AI, weighting values based on the user's health goals). The analysis unit combines these importance scores with the health information itself (e.g., numerical vectors or categorical data for weight, blood pressure, blood glucose, meal contents, amount of exercise) and inputs them into the AI model. The AI model may use a large language model with an importance weighting layer or a multimodal encoder with an attention mechanism to output “analysis detail level label” (e.g., detailed analysis, simple analysis, summary analysis) and “analysis output template selection flag” from the input data. Example inputs include “Blood glucose: importance 0.95, weight: importance 0.60, meal contents: importance 0.80.” The AI model generates outputs such as “Blood glucose: detailed analysis, weight: simple analysis, meal contents: detailed analysis” from these inputs, and the analysis unit presents detailed items such as time-series graphs, anomaly detection results, future prediction values, and risk factor analysis for important items, and displays only averages or trend summaries for general items. Furthermore, the analysis unit can learn from the user's past analysis result viewing history and feedback from medical professionals (e.g., which items were emphasized, which analysis results were referenced) to continuously optimize the level of detail of analysis. As a technical effect, the analysis unit, unlike simple human uniform output or manual settings, achieves essential improvements in computer technology such as suppression of information overload, prevention of missing important information, reduced user burden, and optimized system-wide computational and communication load by performing multidimensional importance analysis and dynamic detail level control with AI models. Specific application fields include home health management systems, patient monitoring in medical institutions, corporate health management support, performance analysis for athletes, and personalized nutrition guidance. Furthermore, by changing the AI model's algorithm or importance score design (e.g., linkage with medical guidelines, linkage with user attributes), flexible system expansion according to user segments and usage environments is possible.

The analysis unit can apply different analysis algorithms according to the category of health information during analysis. The analysis unit applies different analysis algorithms according to the category of health information during analysis, for example. For instance, the analysis unit applies a nutrition analysis algorithm to nutrition information. The analysis unit can apply a health analysis algorithm to health status. Furthermore, the analysis unit can apply the optimal analysis algorithm according to the category of health information. By applying the optimal analysis algorithm according to the category of health information, the analysis unit can obtain more accurate analysis results. Some or all of the above-described processing in the analysis unit may be performed using AI or without using AI. For example, the analysis unit can input the category of health information into generative AI and have the generative AI apply the optimal analysis algorithm. Specifically, the analysis unit accepts input data from the user such as health information category labels (e.g., nutrition information, vital signs, amount of exercise, sleep data, allergy information) and the data itself for each category (e.g., nutrient intake vector, time-series array of heart rate, step count, sleep score, allergy reaction history). The analysis unit applies different preprocessing (e.g., normalization, outlier removal, time-series decomposition, feature extraction) for each category and generates input tensors for the AI model. The AI model may use a multitask learning model with a category selection mechanism or a combination of optimized subnetworks for each category (e.g., regression model for nutrition analysis, time-series prediction model for vital sign analysis, anomaly detection model for allergy reaction detection). Example inputs include “Category: nutrition information, data: protein 60 g, fat 40 g, carbohydrate 200 g” and “Category: vital signs, data: heart rate 70 bpm, blood pressure 120/80 mmHg.” The AI model automatically selects the optimal analysis algorithm for each category from these inputs and outputs structured data such as “nutrition balance evaluation score,” “health risk prediction value,” and “allergy occurrence probability.” Furthermore, the analysis unit can learn from the user's past analysis results and feedback from medical professionals to continuously improve the accuracy of algorithm selection for each category. As a technical effect, the analysis unit, unlike simple human uniform analysis or manual algorithm selection, achieves essential improvements in computer technology such as improved analysis accuracy, maximized computational efficiency, reduced user burden, and increased system-wide flexibility by performing multicategory data analysis and dynamic algorithm optimization with AI models. Specific application fields include home health management systems, multiparameter monitoring in medical institutions, performance analysis for athletes, personalized nutrition guidance, and automatic analysis in telemedicine. Furthermore, by changing the AI model's algorithm configuration or category classification logic (e.g., addition of new categories, branching into subcategories), flexible system expansion according to user segments and usage environments is possible.

The analysis unit can estimate the user's emotion and adjust the length of analysis based on the estimated emotion of the user. The analysis unit estimates the user's emotion and adjusts the length of analysis based on the estimated emotion, for example. For instance, if the user is in a hurry, the analysis unit provides a short analysis result. If the user is relaxed, the analysis unit can provide a detailed analysis result. Furthermore, if the user is feeling stressed, the analysis unit can provide visually easy-to-understand analysis results. By adjusting the length of analysis based on the user's emotion, the analysis unit can provide analysis results suited to the user's situation. Emotion estimation is realized using, for example, an emotion engine or generative AI with emotion estimation functions. Generative AI may be a text generative AI (e.g., LLM) or a multimodal generative AI, but is not limited thereto. Some or all of the above-described processing in the analysis unit may be performed using AI or without using AI. For example, the analysis unit can input the user's emotion data into generative AI and have the generative AI estimate the emotion. Specifically, the analysis unit accepts input data from the user such as audio data (e.g., speech audio spectral tensor, 3 seconds in length, 16 kHz sampling rate), facial expression images (e.g., face image 128×128 pixels RGB tensor), and text chat history (e.g., natural language sentences such as “I'm tired today” or “I'm feeling down”). The analysis unit preprocesses these multimodal data in a preprocessing module by noise removal and normalization, extracts audio features (e.g., MFCC vectorization), image features (e.g., facial landmark detection), and text embeddings (e.g., BERT-based contextual vectorization). The analysis unit concatenates these features into a multidimensional tensor (e.g., audio 128 dimensions+image 256 dimensions+text 768 dimensions=total 1152 dimensions) and inputs it into an emotion estimation AI model. The AI model may use a multimodal Transformer or a combination of convolutional neural networks and recurrent neural networks to output emotion categories (e.g., stress, relaxation, urgency, joy, sadness) and emotion intensity scores (e.g., urgency level 0.80, relaxation level 0.92) from the input data. Example outputs include “Emotion label: urgency, intensity 0.80” and “Emotion label: relaxation, intensity 0.92.” The analysis unit inputs these AI outputs into a threshold judgment module and dynamically switches the length and expression method of analysis results, such as presenting only key points in bullet points if the urgency level is high, generating long analysis results with detailed numerical values and explanatory text if the relaxation level is high, and generating visually easy-to-understand analysis results using graphs or icons if the stress level is high. Furthermore, the analysis unit can refer to the user's past emotion transition history and analysis result viewing history (e.g., time-series vector of emotion scores for the past 7 days, distribution of analysis result viewing times) to learn and adapt the optimal analysis length for each user. As a technical effect, the analysis unit, unlike simple human template switching or uniform output, achieves essential improvements in computer technology such as reduced user burden, improved understanding of analysis results, maximized information transmission efficiency, and optimized system-wide computational and communication load by combining multidimensional emotion analysis with dynamic length optimization using AI models. Specific application fields include home health management systems, analysis reports for patients in telemedicine, corporate health management support, performance analysis for athletes, and mental health support systems. Furthermore, by changing the architecture of the emotion estimation AI model and length optimization algorithms (e.g., user response optimization by reinforcement learning), flexible system expansion according to user segments and usage environments is possible.

The analysis unit can determine the priority of analysis based on the timing of acquiring health information during analysis. The analysis unit determines the priority of analysis based on the timing of acquiring health information during analysis, for example. For instance, the analysis unit preferentially analyzes the latest health information. The analysis unit can determine the priority of analysis by referring to past health information. Furthermore, the analysis unit can adjust the priority of analysis based on the timing of acquiring health information. By determining the priority of analysis based on the timing of acquiring health information, the analysis unit can preferentially analyze the latest information. Some or all of the above-described processing in the analysis unit may be performed using AI or without using AI. For example, the analysis unit can input the timing of acquiring health information into generative AI and have the generative AI determine the priority of analysis. Specifically, the analysis unit accepts input data from the user such as acquisition time stamps for each health information item (e.g., UNIX epoch seconds, date string), data type (e.g., weight, blood pressure, blood glucose, meal contents), and past acquisition history (e.g., array of acquisition times for the past 30 days, distribution of acquisition intervals). The analysis unit preprocesses these time-series data in a preprocessing module by normalization and time-series decomposition, and generates input tensors for the AI model (e.g., multidimensional arrays concatenating [acquisition time, data type, past acquisition interval] for each item). The AI model may use a recurrent neural network for time-series analysis or a large language model with an attention mechanism to output “analysis priority score” and “analysis order label” from the input data. Example inputs include “Weight: 2024-06-01 08:00, blood pressure: 2024-06-01 07:50, blood glucose: 2024-05-31 20:00.” The AI model generates outputs such as “Priority analysis: weight, blood pressure; postpone: blood glucose” from these inputs, and the analysis unit preferentially analyzes the latest data and analyzes past data as supplementary reference information based on the output. Furthermore, the analysis unit can learn from the user's past analysis result viewing history and feedback from medical professionals (e.g., which period's data was emphasized) to continuously optimize the priority of analysis. As a technical effect, the analysis unit, unlike simple human uniform analysis or manual priority setting, achieves essential improvements in computer technology such as maximized information freshness, prevention of missing important information, reduced user burden, and optimized system-wide computational and communication load by performing multidimensional time-point data analysis and dynamic priority control with AI models. Specific application fields include home health management systems, patient monitoring in medical institutions, corporate health management support, performance analysis for athletes, and personalized nutrition guidance. Furthermore, by changing the AI model's algorithm or time-series feature design (e.g., consideration of seasonality and periodicity), flexible system expansion according to user segments and usage environments is possible.

The analysis unit can adjust the order of analysis based on the relevance of health information during analysis. The analysis unit adjusts the order of analysis based on the relevance of health information during analysis, for example. For instance, the analysis unit preferentially analyzes highly relevant health information. The analysis unit can postpone analysis of less relevant health information. Furthermore, the analysis unit can adjust the order of analysis based on the relevance of health information. By adjusting the order of analysis based on the relevance of health information, the analysis unit can preferentially analyze highly relevant information. Some or all of the above-described processing in the analysis unit may be performed using AI or without using AI. For example, the analysis unit can input the relevance of health information into generative AI and have the generative AI adjust the order of analysis. Specifically, the analysis unit accepts input data from the user such as relevance scores between health information items (e.g., correlation coefficients, relevance labels based on medical guidelines, automatic estimation values by AI) and the data itself for each item (e.g., numerical vectors or categorical data for weight, blood pressure, blood glucose, meal contents). The analysis unit represents these relevance scores as graph structure data (e.g., nodes=health information items, edges=relevance weights) and generates input tensors for the AI model. The AI model may use a graph neural network or a large language model with an attention mechanism to output “analysis order label” and “priority analysis item list” from the input data. Example inputs include “Weight-blood pressure: relevance 0.85, blood pressure-blood glucose: relevance 0.60.” The AI model generates outputs such as “Priority analysis: weight, blood pressure; postpone: blood glucose” from these inputs, and the analysis unit preferentially analyzes highly relevant items and postpones less relevant items based on the output. Furthermore, the analysis unit can learn from the user's past analysis result viewing history and feedback from medical professionals (e.g., which item combinations were emphasized) to continuously optimize the order of analysis. As a technical effect, the analysis unit, unlike simple human uniform analysis or manual order setting, achieves essential improvements in computer technology such as maximized information transmission efficiency, prevention of missing important information, reduced user burden, and optimized system-wide computational and communication load by performing multidimensional relevance analysis and dynamic order control with AI models. Specific application fields include home health management systems, multiparameter monitoring in medical institutions, corporate health management support, performance analysis for athletes, and personalized nutrition guidance. Furthermore, by changing the AI model's algorithm or relevance score design (e.g., linkage with medical knowledge graphs, linkage with user attributes), flexible system expansion according to user segments and usage environments is possible.

The provision unit can estimate the user's emotion and adjust the expression method of provision based on the estimated emotion of the user. The provision unit estimates the user's emotion and adjusts the expression method of provision based on the estimated emotion, for example. For instance, if the user is relaxed, the provision unit displays detailed provision contents. If the user is in a hurry, the provision unit can display concise provision contents. Furthermore, if the user is feeling stressed, the provision unit can display visually easy-to-understand provision contents. By adjusting the expression method of provision based on the user's emotion, the provision unit can display more appropriate provision contents. Emotion estimation is realized using, for example, an emotion engine or generative AI with emotion estimation functions. Generative AI may be a text generative AI (e.g., LLM) or a multimodal generative AI, but is not limited thereto. Some or all of the above-described processing in the provision unit may be performed using AI or without using AI. For example, the provision unit can input the user's emotion data into generative AI and have the generative AI estimate the emotion. Specifically, the provision unit accepts input data from the user such as audio data (e.g., speech audio spectral tensor, 3 seconds in length, 16 kHz sampling rate), facial expression images (e.g., face image 128×128 pixels RGB tensor), and text chat history (e.g., natural language sentences such as “I'm tired today” or “I'm feeling down”). The provision unit preprocesses these multimodal data in a preprocessing module by noise removal and normalization, extracts audio features (e.g., MFCC vectorization), image features (e.g., facial landmark detection), and text embeddings (e.g., BERT-based contextual vectorization). The provision unit concatenates these features into a multidimensional tensor (e.g., audio 128 dimensions+image 256 dimensions+text 768 dimensions=total 1152 dimensions) and inputs it into an emotion estimation AI model. The AI model may use a multimodal Transformer or a combination of convolutional neural networks and recurrent neural networks to output emotion categories (e.g., stress, relaxation, urgency, joy, sadness) and emotion intensity scores (e.g., stress level 0.85, relaxation level 0.10) from the input data. Example outputs include “Emotion label: stress, intensity 0.78” and “Emotion label: relaxation, intensity 0.92.” The provision unit inputs these AI outputs into a threshold judgment module and dynamically switches the expression method, such as generating visually easy-to-understand provision contents using graphs or icons if the stress level is high, generating provision contents with detailed numerical values and explanatory text if the relaxation level is high, and presenting only key points in bullet points if the urgency level is high. Furthermore, the provision unit can refer to the user's past emotion transition history and provision content viewing history (e.g., time-series vector of emotion scores for the past 7 days, distribution of provision content viewing times) to learn and adapt the optimal expression style for each user. As a technical effect, the provision unit, unlike simple human template switching or uniform output, achieves essential improvements in computer technology such as reduced user burden, improved understanding of provision contents, maximized information transmission efficiency, and optimized system-wide computational and communication load by combining multidimensional emotion analysis with dynamic expression optimization using AI models. Specific application fields include home health management systems, meal proposal reports for patients in telemedicine, corporate health management support, performance meal proposals for athletes, and mental health support systems. Furthermore, by changing the architecture of the emotion estimation AI model and expression optimization algorithms (e.g., user response optimization by reinforcement learning), flexible system expansion according to user segments and usage environments is possible.

The provision unit can adjust the level of detail of provision based on the importance of meal contents at the time of provision. For example, the provision unit adjusts the level of detail of provision according to the importance of meal contents at the time of provision. For instance, the provision unit provides detailed information for important meal contents. Additionally, the provision unit can provide concise information for general meal contents. Furthermore, the provision unit can adjust the level of detail of provision based on the importance of meal contents. By adjusting the level of detail of provision according to the importance of meal contents, the provision unit can provide important information in detail. Some or all of the above-described processing in the provision unit may be performed using AI, or may be performed without using AI. For example, the provision unit can input the importance of meal contents to a generative AI and have the generative AI execute the adjustment of the level of detail of provision. Specifically, the provision unit accepts, as input data from the user, importance scores for each meal content (e.g., priority labels set by doctors or registered dietitians, automatic scoring values by AI, weighting values based on the user's health goals). The provision unit combines these importance scores with the main body of meal contents (e.g., dish name, estimated nutrient amount, allergy risk score, satisfaction prediction score, etc. as structured data) and inputs them to an AI model. As the AI model, a large language model with an importance weighting layer or a multimodal encoder with an attention mechanism is used to output a “provision detail label” (e.g., detailed provision, simplified provision, summary provision, etc.) and a “provision output template selection flag” from the input data. Examples of input include “Grilled fish set meal: importance 0.95, vegetable salad: importance 0.60, curry rice: importance 0.80”. The AI model generates outputs such as “Grilled fish set meal: detailed provision, vegetable salad: simplified provision, curry rice: detailed provision” from these inputs, and the provision unit, based on the output, presents detailed information such as nutrient composition graphs, allergy precautions, and cooking process details for important items, and displays only the dish name and major nutrients for general items. Furthermore, the provision unit can continuously optimize the level of detail of provision automatically by learning from the user's past provision content viewing history and feedback from medical professionals (e.g., which items were emphasized, which provision contents were referenced). As a technical effect, the provision unit, unlike simple uniform output or manual settings by humans, achieves essential improvements in computer technology such as suppression of information overload, prevention of missing important information, reduction of user burden, and optimization of overall system computation and communication load through multidimensional importance analysis and dynamic detail control by AI models. Specific application fields include home health management systems, patient meal proposals in medical institutions, corporate health management support, performance meal management for athletes, and personalized nutrition guidance. Furthermore, by changing the algorithms of the AI model and the design of importance scores (e.g., linkage with medical guidelines, linkage with user attributes), flexible system expansion according to user groups and usage environments is possible.

The provision unit can apply different provision algorithms according to the category of meal contents at the time of provision. For example, the provision unit applies different provision algorithms according to the category of meal contents at the time of provision. For instance, the provision unit applies a nutrition provision algorithm to nutritional information. Additionally, the provision unit can apply a health provision algorithm to health status. Furthermore, the provision unit can apply the optimal provision algorithm according to the category of meal contents. By applying the optimal provision algorithm according to the category of meal contents, the provision unit can display more accurate provision contents. Some or all of the above-described processing in the provision unit may be performed using AI, or may be performed without using AI. For example, the provision unit can input the category of meal contents to a generative AI and have the generative AI execute the application of the optimal provision algorithm. Specifically, the provision unit accepts, as input data from the user, meal content category labels (e.g., nutritional information, vital signs, allergy information, preference information, etc.) and the data body for each category (e.g., nutrient intake vector, allergy risk score, preference cluster label, etc.). The provision unit applies different preprocessing for each category (e.g., normalization, outlier removal, feature extraction) and generates input tensors for the AI model. As the AI model, a multitask learning model with a category selection mechanism or a combination of optimized subnetworks for each category (e.g., regression model for nutrition provision, anomaly detection model for allergy caution provision, etc.) is used. Examples of input include “Category: nutritional information, Data: protein 60 g, fat 40 g, carbohydrate 200 g” and “Category: allergy information, Data: high risk for shrimp”. The AI model automatically selects the optimal provision algorithm for each category from these inputs and generates structured data such as “nutritional balance evaluation graph” and “allergy caution label” as output. Furthermore, the provision unit can continuously improve the accuracy of algorithm selection for each category by learning from the user's past provision contents and feedback from medical professionals. As a technical effect, the provision unit, unlike simple uniform provision or manual algorithm selection by humans, achieves essential improvements in computer technology such as improvement of provision accuracy, maximization of computational efficiency, reduction of user burden, and enhancement of overall system flexibility through multicategory data analysis and dynamic algorithm optimization by AI models. Specific application fields include home health management systems, multi-item meal proposals in medical institutions, performance meal management for athletes, personalized nutrition guidance, and automatic meal proposals in remote medical care. Furthermore, by changing the algorithm configuration of the AI model and the category classification logic (e.g., addition of new categories, branching of subcategories), flexible system expansion according to user groups and usage environments is possible.

The provision unit can estimate the user's emotion and adjust the length of provision based on the estimated emotion of the user. For example, the provision unit estimates the user's emotion and adjusts the length of provision based on the estimated emotion. For instance, if the user is in a hurry, the provision unit displays short provision contents. Additionally, if the user is relaxed, the provision unit can display detailed provision contents. Furthermore, if the user is feeling stressed, the provision unit can display provision contents that are visually easy to understand. By adjusting the length of provision based on the user's emotion, the provision unit can display provision contents suitable for the user's situation. Emotion estimation is realized, for example, by using an emotion engine or a generative AI with emotion estimation functionality. The generative AI may be a text generation AI (e.g., LLM) or a multimodal generative AI, but is not limited to such examples. Some or all of the above-described processing in the provision unit may be performed using AI, or may be performed without using AI. For example, the provision unit can input the user's emotion data to a generative AI and have the generative AI execute emotion estimation. Specifically, the provision unit accepts, as input data from the user, voice data (e.g., speech audio spectrum tensor, 3 seconds in length, 16 kHz sampling rate), facial expression images (e.g., face image 128×128 pixels RGB tensor), and text chat history (e.g., natural language sentences such as “I'm tired today”, “I'm feeling down”). The provision unit preprocesses these multimodal data with a preprocessing module for noise removal and normalization, extracts audio features (e.g., MFCC vectorization), image features (e.g., facial landmark detection), and text embeddings (e.g., BERT-based contextual vectorization). The provision unit concatenates these features into a multidimensional tensor (e.g., audio 128 dimensions+image 256 dimensions+text 768 dimensions=total 1152 dimensions) and inputs it to an emotion estimation AI model. As the AI model, a multimodal Transformer or a combination of convolutional neural networks and recurrent neural networks is used to output emotion categories (e.g., stress, relaxation, hurry, joy, sadness, etc.) and emotion intensity scores (e.g., stress level 0.85, relaxation level 0.10, etc.) from the input data. Examples of output include “Emotion label: hurry, intensity 0.80” and “Emotion label: relaxation, intensity 0.92”. The provision unit inputs these AI outputs to a threshold judgment module, and if the hurry level is high, presents only the main points in bullet form; if the relaxation level is high, generates long provision contents with detailed numerical values and explanatory text; and if the stress level is high, generates visually easy-to-understand provision contents using graphs and icons, dynamically switching the length and expression method of provision contents. Furthermore, the provision unit can refer to the user's past emotion transition history and provision content viewing history (e.g., time series vector of emotion scores over the past 7 days, distribution of viewing times for provision contents) to learn and adapt the optimal provision length for each user. As a technical effect, the provision unit, unlike simple template switching or uniform output by humans, achieves essential improvements in computer technology such as reduction of user burden, improvement of understanding of provision contents, maximization of information transmission efficiency, and optimization of overall system computation and communication load by combining multidimensional emotion analysis and dynamic length optimization by AI models. Specific application fields include home health management systems, patient meal proposal reports in remote medical care, corporate health management support, performance meal proposals for athletes, and mental health support systems. Furthermore, by changing the architecture of the emotion estimation AI model and the length optimization algorithm (e.g., user response optimization by reinforcement learning), flexible system expansion according to user groups and usage environments is possible.

The provision unit can determine the priority of provision based on the timing of submission of meal contents at the time of provision. For example, the provision unit determines the priority of provision based on the timing of submission of meal contents at the time of provision. For instance, the provision unit preferentially provides the latest meal contents. Additionally, the provision unit can determine the priority of provision by referring to past meal contents. Furthermore, the provision unit can adjust the priority of provision based on the timing of submission of meal contents. By determining the priority of provision based on the timing of submission of meal contents, the provision unit can preferentially provide the latest information. Some or all of the above-described processing in the provision unit may be performed using AI, or may be performed without using AI. For example, the provision unit can input the timing of submission of meal contents to a generative AI and have the generative AI execute the determination of provision priority. Specifically, the provision unit accepts, as input data from the user, submission time stamps for each meal content (e.g., UNIX epoch seconds, date strings), data types (e.g., main dish, side dish, dessert, etc.), and past submission history (e.g., array of submission times for the past 30 days, distribution of submission intervals). The provision unit normalizes and decomposes these time series data with a preprocessing module and generates input tensors for the AI model (e.g., multidimensional arrays concatenating [submission time, data type, past submission interval] for each item). As the AI model, a recurrent neural network for time series analysis or a large language model with an attention mechanism is used to output “provision priority score” and “provision order label” from the input data. Examples of input include “Main dish: 2024-06-01 08:00, Side dish: 2024-06-01 07:50, Dessert: 2024-05-31 20:00”. The AI model generates outputs such as “Priority provision: main dish and side dish, deferred: dessert” from these inputs, and the provision unit, based on the output, preferentially provides the latest data and provides past data as supplementary reference information. Furthermore, the provision unit can continuously optimize the priority of provision automatically by learning from the user's past provision content viewing history and feedback from medical professionals (e.g., which period's data was emphasized). As a technical effect, the provision unit, unlike simple uniform provision or manual priority setting by humans, achieves essential improvements in computer technology such as maximization of information freshness, prevention of missing important information, reduction of user burden, and optimization of overall system computation and communication load through multi-time-point data analysis and dynamic priority control by AI models. Specific application fields include home health management systems, patient meal proposals in medical institutions, corporate health management support, performance meal management for athletes, and personalized nutrition guidance. Furthermore, by changing the algorithms of the AI model and the design of time series features (e.g., consideration of seasonality and periodicity), flexible system expansion according to user groups and usage environments is possible.

The provision unit can adjust the order of provision based on the relevance of meal contents at the time of provision. For example, the provision unit adjusts the order of provision based on the relevance of meal contents at the time of provision. For instance, the provision unit preferentially provides highly relevant meal contents. Additionally, the provision unit can defer the provision of less relevant meal contents. Furthermore, the provision unit can adjust the order of provision based on the relevance of meal contents. By adjusting the order of provision based on the relevance of meal contents, the provision unit can preferentially provide highly relevant information. Some or all of the above-described processing in the provision unit may be performed using AI, or may be performed without using AI. For example, the provision unit can input the relevance of meal contents to a generative AI and have the generative AI execute the adjustment of provision order. Specifically, the provision unit accepts, as input data from the user, relevance scores between meal content items (e.g., nutrient interaction coefficients, relevance labels based on medical guidelines, automatic estimation values by AI), and the data body for each item (e.g., category data such as main dish, side dish, dessert, or nutrient vectors). The provision unit represents these relevance scores as graph structure data (e.g., nodes=meal content items, edges=relevance weights) and generates input tensors for the AI model. As the AI model, a graph neural network or a large language model with an attention mechanism is used to output “provision order label” and “priority provision item list” from the input data. Examples of input include “Main dish-side dish: relevance 0.85, side dish-dessert: relevance 0.60”. The AI model generates outputs such as “Priority provision: main dish and side dish, deferred: dessert” from these inputs, and the provision unit, based on the output, preferentially provides highly relevant items and defers less relevant items. Furthermore, the provision unit can continuously optimize the order of provision automatically by learning from the user's past provision content viewing history and feedback from medical professionals (e.g., which combinations of items were emphasized). As a technical effect, the provision unit, unlike simple uniform provision or manual order setting by humans, achieves essential improvements in computer technology such as maximization of information transmission efficiency, prevention of missing important information, reduction of user burden, and optimization of overall system computation and communication load through multi-item relevance analysis and dynamic order control by AI models. Specific application fields include home health management systems, multi-item meal proposals in medical institutions, corporate health management support, performance meal management for athletes, and personalized nutrition guidance. Furthermore, by changing the algorithms of the AI model and the design of relevance scores (e.g., linkage with medical knowledge graphs, linkage with user attributes), flexible system expansion according to user groups and usage environments is possible.

The delivery unit can estimate the user's emotion and adjust the delivery method based on the estimated emotion of the user. For example, the delivery unit estimates the user's emotion and adjusts the delivery method based on the estimated emotion. For instance, if the user is relaxed, the delivery unit uses the normal delivery method. Additionally, if the user is in a hurry, the delivery unit can use a rapid delivery method. Furthermore, if the user is feeling stressed, the delivery unit can shorten the delivery time. By adjusting the delivery method based on the user's emotion, the delivery unit can select a more appropriate delivery method. Emotion estimation is realized, for example, by using an emotion engine or a generative AI with emotion estimation functionality. The generative AI may be a text generation AI (e.g., LLM) or a multimodal generative AI, but is not limited to such examples. Some or all of the above-described processing in the delivery unit may be performed using AI, or may be performed without using AI. For example, the delivery unit can input the user's emotion data to a generative AI and have the generative AI execute emotion estimation. Specifically, the delivery unit accepts, as input data from the user, voice data (e.g., speech audio spectrum tensor, 3 seconds in length, 16 kHz sampling rate), facial expression images (e.g., face image 128×128 pixels RGB tensor), and text chat history (e.g., natural language sentences such as “I'm tired today”, “I'm in a hurry”). The delivery unit preprocesses these multimodal data with a preprocessing module for noise removal and normalization, extracts audio features (e.g., MFCC vectorization), image features (e.g., facial landmark detection), and text embeddings (e.g., BERT-based contextual vectorization). The delivery unit concatenates these features into a multidimensional tensor (e.g., audio 128 dimensions+image 256 dimensions+text 768 dimensions=total 1152 dimensions) and inputs it to an emotion estimation AI model. As the AI model, a multimodal Transformer or a combination of convolutional neural networks and recurrent neural networks is used to output emotion categories (e.g., stress, relaxation, hurry, joy, sadness, etc.) and emotion intensity scores (e.g., stress level 0.85, relaxation level 0.10, etc.) from the input data. Examples of output include “Emotion label: hurry, intensity 0.80” and “Emotion label: relaxation, intensity 0.92”. The delivery unit inputs these AI outputs to a threshold judgment module, and if the hurry level is high, prioritizes the shortest route and fastest means (e.g., drone, motorcycle); if the relaxation level is high, selects the normal route and standard means (e.g., vehicle, walking); and if the stress level is high, automatically applies special measures such as shortened delivery time or advance notification to the delivery person. Furthermore, the delivery unit can continuously improve the optimization accuracy of delivery methods by learning from the user's past emotion transition history and delivery satisfaction scores. As a technical effect, the delivery unit, unlike simple delivery arrangements or uniform rules by humans, achieves essential improvements in computer technology such as maximization of user satisfaction, shortening of delivery time, reduction of delivery errors, and optimization of overall system computation and communication load by combining multidimensional emotion analysis and dynamic delivery method optimization by AI models. Specific application fields include meal delivery for homebound elderly, corporate welfare services, performance meal delivery for athletes, patient meal supply in remote medical care, and mental health support systems. Furthermore, by changing the architecture of the emotion estimation AI model and the delivery method optimization algorithm (e.g., user response optimization by reinforcement learning), flexible system expansion according to user groups and usage environments is possible.

The delivery unit can analyze the user's past delivery history and select the optimal delivery method at the time of delivery. For example, the delivery unit analyzes the user's past delivery history and selects the optimal delivery method at the time of delivery. For instance, the delivery unit selects the optimal delivery method based on the delivery methods previously used by the user. Additionally, the delivery unit can select a rapid delivery method from the user's past delivery history. Furthermore, the delivery unit can analyze the user's past delivery history and select the most efficient delivery method. By analyzing the user's past delivery history, the delivery unit can select the optimal delivery method. Some or all of the above-described processing in the delivery unit may be performed using AI, or may be performed without using AI. For example, the delivery unit can input the user's past delivery history to a generative AI and have the generative AI execute the selection of the optimal delivery method. Specifically, the delivery unit collects delivery history data for each user over the past year (e.g., delivery means ID, delivery date and time, delivery duration, delivery success rate, user satisfaction score, delivery person ID, delivery cost, etc. as a structured table). The delivery unit inputs these history data as time series vectors or category label arrays (e.g., 10 drone deliveries, 20 motorcycle deliveries, 5 vehicle deliveries, etc.) to the AI model. As the AI model, a recurrent neural network for time series analysis or a decision tree-based gradient boosting model is used to output effectiveness scores for each delivery means (e.g., duration 0.95, cost 0.80, satisfaction 0.90, etc.) and optimal delivery timing (e.g., 7 a.m., 9 p.m., etc.) from the input data. Examples of input include “Past 30 days: drone delivery success rate 98%, motorcycle delivery average duration 15 minutes, vehicle delivery satisfaction score 0.92”. The AI model generates outputs such as “Recommended delivery means: drone, recommended delivery time: 7 a.m., predicted satisfaction 0.97” from these inputs. The delivery unit automatically selects the optimal delivery means and timing for each user based on the AI output and dynamically changes the system settings. Furthermore, when the delivery unit detects changes in the user's lifestyle (e.g., introduction of new delivery means, change in daily rhythm), it can perform relearning or reevaluation and continuously optimize the delivery method. As a technical effect, the delivery unit, unlike human heuristics or uniform settings, achieves essential improvements in computer technology such as improvement of delivery accuracy, reduction of user burden, minimization of delivery failure rate, and reduction of system operation costs through history data analysis and dynamic optimization by AI models. Specific application fields include home health management systems, corporate welfare services, patient meal delivery in medical institutions, performance meal management for athletes, and delivery optimization in remote medical care. Furthermore, by changing the algorithms and feature design of the AI model (e.g., addition of user attributes, delivery means performance, environmental factors) and the delivery method selection logic (e.g., dynamic optimization by multi-armed bandit), flexible system expansion according to user groups and usage environments is possible.

The delivery unit can customize the means of delivery based on the user's current lifestyle at the time of delivery. For example, the delivery unit customizes the means of delivery based on the user's current lifestyle at the time of delivery. For instance, if the user is at work, the delivery unit adjusts the delivery time. Additionally, if the user is on vacation, the delivery unit can flexibly set the delivery time. Furthermore, the delivery unit can select the optimal means of delivery based on the user's lifestyle. By customizing the means of delivery based on the user's current lifestyle, the delivery unit can select a more appropriate means of delivery. Some or all of the above-described processing in the delivery unit may be performed using AI, or may be performed without using AI. For example, the delivery unit can input the user's current lifestyle to a generative AI and have the generative AI execute the customization of the means of delivery. Specifically, the delivery unit accepts, as input data from the user, lifestyle information (e.g., occupation category, working hours, holiday information, travel flag, at-home/outdoor status as category data), current activity status (e.g., in a meeting, exercising, resting as status labels), and delivery preferences (e.g., contactless receipt, leave at the door, time specification, etc.). The delivery unit encodes this information using one-hot encoding or embedding vectors and combines it with a list of candidate delivery means (e.g., drone, motorcycle, vehicle, walking as item arrays) to input to the AI model. As the AI model, a combination of rule-based filtering and large language models or a multimodal encoder is used to output “delivery means selection score” and “delivery time adjustment flag” from the input data. Examples of input include “Lifestyle: at work, Activity: in a meeting, Preference: contactless receipt”. The AI model generates outputs such as “Recommended delivery means: leave at the door, recommended delivery time: lunch break” from these inputs. The delivery unit, based on the AI output, suppresses unnecessary delivery means and inappropriate delivery times, and efficiently selects only the necessary means and times. Furthermore, the delivery unit can continuously improve customization accuracy by learning from the user's past delivery customization history and delivery results (e.g., delivery success rate, user satisfaction score). As a technical effect, the delivery unit, unlike simple item selection or uniform delivery by humans, achieves essential improvements in computer technology such as reduction of delivery noise, minimization of user burden, optimization of overall system computation and communication load, and improvement of delivery service utility through multidimensional data analysis and dynamic customization by AI models. Specific application fields include home health management, corporate welfare services, performance meal delivery for athletes, patient meal supply in remote medical care, and personalized delivery services. Furthermore, by changing the algorithms and feature design of the AI model (e.g., addition of lifestyle, activity, and environmental factors) and the customization logic (e.g., optimization by reinforcement learning), flexible system expansion according to user groups and usage environments is possible.

The delivery unit can estimate the user's emotion and determine the priority of delivery based on the estimated emotion of the user. For example, the delivery unit estimates the user's emotion and determines the priority of delivery based on the estimated emotion. For instance, if the user is in a hurry, the delivery unit sets a high priority for delivery. Additionally, if the user is relaxed, the delivery unit can set the normal delivery priority. Furthermore, if the user is feeling stressed, the delivery unit can adjust the priority of delivery. By determining the priority of delivery based on the user's emotion, the delivery unit can preferentially perform important deliveries. Emotion estimation is realized, for example, by using an emotion engine or a generative AI with emotion estimation functionality. The generative AI may be a text generation AI (e.g., LLM) or a multimodal generative AI, but is not limited to such examples. Some or all of the above-described processing in the delivery unit may be performed using AI, or may be performed without using AI. For example, the delivery unit can input the user's emotion data to a generative AI and have the generative AI execute emotion estimation. Specifically, the delivery unit accepts, as input data from the user, voice data (e.g., speech audio spectrum tensor, 3 seconds in length, 16 kHz sampling rate), facial expression images (e.g., face image 128×128 pixels RGB tensor), and text chat history (e.g., natural language sentences such as “I'm in a hurry today”, “High stress”). The delivery unit preprocesses these multimodal data with a preprocessing module for noise removal and normalization, extracts audio features (e.g., MFCC vectorization), image features (e.g., facial landmark detection), and text embeddings (e.g., BERT-based contextual vectorization). The delivery unit concatenates these features into a multidimensional tensor (e.g., audio 128 dimensions+image 256 dimensions+text 768 dimensions=total 1152 dimensions) and inputs it to an emotion estimation AI model. As the AI model, a multimodal Transformer or a combination of convolutional neural networks and recurrent neural networks is used to output emotion categories (e.g., stress, relaxation, hurry, etc.) and emotion intensity scores (e.g., hurry level 0.90, stress level 0.80, etc.) from the input data. Examples of output include “Emotion label: hurry, intensity 0.85” and “Emotion label: relaxation, intensity 0.92”. The delivery unit inputs these AI outputs to a threshold judgment module, and if the hurry level is high, sets the delivery priority to the highest; if the stress level is high, applies special measures (e.g., advance contact by the delivery person, shortened delivery time); and if the relaxation level is high, maintains the normal priority. Furthermore, the delivery unit can continuously improve the accuracy of priority determination by learning from the user's past emotion transition history and delivery results (e.g., delivery satisfaction score, redelivery rate). As a technical effect, the delivery unit, unlike simple uniform priority setting or manual adjustment by humans, achieves essential improvements in computer technology such as prevention of missing important deliveries, maximization of user satisfaction, improvement of delivery efficiency, and optimization of overall system computation and communication load through multidimensional emotion analysis and dynamic priority control by AI models. Specific application fields include home health management systems, corporate welfare services, performance meal delivery for athletes, patient meal supply in remote medical care, and mental health support systems. Furthermore, by changing the architecture of the emotion estimation AI model and the priority determination algorithm (e.g., optimization by reinforcement learning), flexible system expansion according to user groups and usage environments is possible.

The delivery unit can select the optimal delivery method by considering the user's geographic location information at the time of delivery. For example, the delivery unit selects the optimal delivery method by considering the user's geographic location information at the time of delivery. For instance, if the user is in a remote location, the delivery unit selects the optimal delivery method. Additionally, the delivery unit can select a rapid delivery method based on the user's geographic location information. Furthermore, the delivery unit can adjust the means of delivery by considering the user's geographic location information. By considering the user's geographic location information, the delivery unit can select the optimal delivery method. Some or all of the above-described processing in the delivery unit may be performed using AI, or may be performed without using AI. For example, the delivery unit can input the user's geographic location information to a generative AI and have the generative AI execute the selection of the optimal delivery method. Specifically, the delivery unit accepts, as input data from the user, geographic location information (e.g., GPS coordinate vector, address string, region ID), environmental information of the current location (e.g., traffic congestion level, weather, road conditions, building type, floor number), and operational status of delivery means (e.g., drone battery level, current location of motorcycle, number of vehicles). The delivery unit normalizes and standardizes this information with a preprocessing module and generates geographic feature vectors (e.g., latitude, longitude, elevation, region cluster ID, etc.). The delivery unit combines these with a list of candidate delivery means (e.g., drone, motorcycle, vehicle, walking) and inputs them to the AI model. As the AI model, a large language model with a geographic information embedding layer or a graph neural network is used to output “delivery means selection score” and “recommended delivery route label” from the input data. Examples of input include “Current location: Shinjuku, Tokyo; high traffic congestion; weather: rain; drone battery 80%”. The AI model generates outputs such as “Recommended delivery means: motorcycle; recommended route: A” from these inputs, and the delivery unit automatically selects the optimal delivery means and route according to geographic conditions based on the output. Furthermore, the delivery unit can continuously improve the accuracy of delivery method selection by learning from the user's past geographic movement history and delivery results (e.g., delivery success rate, user satisfaction score). As a technical effect, the delivery unit, unlike simple item selection or uniform delivery by humans, achieves essential improvements in computer technology such as maximization of delivery efficiency, minimization of user burden, optimization of overall system computation and communication load, and improvement of delivery service utility through multidimensional geographic information analysis and dynamic delivery means optimization by AI models. Specific application fields include home health management, corporate welfare services, performance meal delivery for athletes, patient meal supply in remote medical care, and region-specific delivery optimization. Furthermore, by changing the algorithms and feature design of the AI model (e.g., addition of geographic and environmental factors) and the delivery means selection logic (e.g., optimization by reinforcement learning), flexible system expansion according to user groups and usage environments is possible.

The delivery unit can analyze the user's social media activity and propose means of delivery at the time of delivery. For example, the delivery unit analyzes the user's social media activity and proposes means of delivery at the time of delivery. For instance, the delivery unit analyzes requests related to delivery from the user's social media activity and proposes the optimal means. Additionally, the delivery unit can select means of delivery based on the user's social media activity. Furthermore, the delivery unit can analyze the user's social media activity and customize the means of delivery. By analyzing the user's social media activity, the delivery unit can propose the optimal means of delivery. Some or all of the above-described processing in the delivery unit may be performed using AI, or may be performed without using AI. For example, the delivery unit can input the user's social media activity to a generative AI and have the generative AI execute the proposal of means of delivery. Specifically, the delivery unit accepts, as input data from the user, social media post data (e.g., text posts, image posts, video posts, posting time, hashtags, number of likes, number of comments, etc. as structured data), requests and feedback related to delivery (e.g., natural language sentences such as “Request drone delivery”, “Request contactless receipt”), and follow relationships and trend information (e.g., delivery service ID, delivery means trend label). The delivery unit preprocesses these data with a natural language processing module or image analysis module, generates text embedding vectors (e.g., BERT-based contextual vectors), image feature vectors (e.g., ResNet-based image embeddings), and post metadata vectors (e.g., posting frequency, time distribution, etc.). The delivery unit concatenates these features into a multidimensional tensor (e.g., text 768 dimensions+image 256 dimensions+metadata 32 dimensions=total 1056 dimensions) and inputs it to the AI model. As the AI model, a multimodal Transformer or a graph neural network is used to output extraction of means of delivery, estimation of delivery requests, delivery means selection score, customization flag, etc. from the input data. Examples of input include “Recent post: ‘Drone delivery is convenient’, ‘Request contactless receipt’; image: delivery box photo; hashtag: #delivery trend”. The AI model generates outputs such as “Recommended means of delivery: drone; customization: contactless receipt” from these inputs. The delivery unit, based on the AI output, suppresses unnecessary delivery means proposals and preferentially proposes means related to the user's interests and requests. Furthermore, the delivery unit can continuously improve the accuracy of proposals and customization by learning from the user's past social media activity history and delivery results (e.g., delivery success rate, user satisfaction score). As a technical effect, the delivery unit, unlike simple item selection or uniform delivery by humans, achieves essential improvements in computer technology such as reduction of delivery noise, minimization of user burden, optimization of overall system computation and communication load, and improvement of delivery service utility through multidimensional social data analysis and dynamic proposal of means of delivery by AI models. Specific application fields include home health management, corporate welfare services, performance meal delivery for athletes, patient meal supply in remote medical care, and health trend analysis-based delivery services. Furthermore, by changing the algorithms and feature design of the AI model (e.g., addition of post content, images, trend factors) and the delivery means proposal logic (e.g., optimization by reinforcement learning), flexible system expansion according to user groups and usage environments is possible.

The system according to the embodiment is not limited to the above-described examples, and various modifications are possible, for example, as follows. Specifically, the present system has extensibility that allows flexible changes to the AI model configuration, data flow, and algorithm choices for each unit. By designing the interface specifications between the acquisition unit, analysis unit, provision unit, and delivery unit modules based on APIs, the system can easily cooperate with different AI architectures (e.g., Transformer, graph neural networks, convolutional neural networks, etc.) and heterogeneous data sources (e.g., wearable devices, smartphone applications, cloud health management services, etc.). By changing the learning methods of the AI model (e.g., transfer learning, multitask learning, self-supervised learning, reinforcement learning) and feature design (e.g., addition of user attributes, environmental factors, time series variations, social factors), the system can be optimized according to user groups, usage environments, regional characteristics, and disease types. The system can expand the delivery means management module of the delivery unit to cooperate with new delivery infrastructures such as drones, autonomous vehicles, and robots, and implement dynamic route optimization considering real-time traffic information, weather information, and disaster information. Furthermore, the system can incorporate an adaptive UI generation function that automatically switches the output format of the analysis unit and provision unit according to user attributes and the terminal used (e.g., smartphone, tablet, smart speaker, etc.). As a technical effect, the present system, unlike conventional health management systems with single-purpose and fixed configurations, can flexibly apply various combinations of AI models, data flows, interfaces, and algorithms, thereby greatly improving system extensibility, maintainability, and adaptability, maximizing personalization for each user, reducing operational costs, and enabling prompt response to future new technology introduction, thus achieving essential improvements in computer technology. Specific application fields include home health management, corporate health management support, patient monitoring in medical institutions, performance management for athletes, remote medical care, health and meal supply support during disasters, region-specific health risk management, and personalized nutrition guidance. Furthermore, by changing the API linkage specifications and data expansion methods of the AI model, future cooperation with IoT devices, new sensors, and external health databases can be easily realized.

The analysis unit can estimate the user's emotion and adjust the proposal of meal contents based on the estimated emotion of the user. For example, if the user is feeling stressed, the analysis unit proposes menus containing ingredients with relaxation effects. Additionally, if the user is joyful, the analysis unit can propose special desserts. Furthermore, if the user is tired, the analysis unit can propose meals suitable for energy replenishment. By adjusting the proposal of meal contents based on the user's emotion, the analysis unit can provide meals that are attentive to the user's emotion. Specifically, the analysis unit accepts, as input data from the user, voice data (e.g., speech audio spectrum tensor, 3 seconds in length, 16 kHz sampling rate), facial expression images (e.g., face image 128×128 pixels RGB tensor), and text chat history (e.g., natural language sentences such as “I'm tired today”, “High stress”). The analysis unit preprocesses these multimodal data with a preprocessing module for noise removal and normalization, extracts audio features (e.g., MFCC vectorization), image features (e.g., facial landmark detection), and text embeddings (e.g., BERT-based contextual vectorization). The analysis unit concatenates these features into a multidimensional tensor (e.g., audio 128 dimensions+image 256 dimensions+text 768 dimensions=total 1152 dimensions) and inputs it to an emotion estimation AI model. As the AI model, a multimodal Transformer or a combination of convolutional neural networks and recurrent neural networks is used to output emotion categories (e.g., stress, relaxation, fatigue, joy, etc.) and emotion intensity scores (e.g., stress level 0.85, relaxation level 0.10, etc.) from the input data. Examples of output include “Emotion label: stress, intensity 0.78” and “Emotion label: fatigue, intensity 0.82”. The analysis unit inputs these AI outputs to a threshold judgment module, and if the stress level is high, preferentially proposes menus containing ingredients with relaxation effects (e.g., herbal tea, seafood, dairy products, etc.); if the fatigue level is high, proposes high-protein, high-carbohydrate menus suitable for energy replenishment (e.g., chicken dishes, pasta, etc.); and if the joy level is high, proposes special desserts or reward menus (e.g., fruit parfait, cake, etc.). The analysis unit can also refer to the user's past emotion transition history and meal history (e.g., time series vector of emotion scores over the past 7 days, meal content history) and preferentially re-propose menus that were well received during similar emotional states, thereby enhancing personalization. As a technical effect, the analysis unit, unlike simple template proposals or uniform menu presentation by humans, achieves essential improvements in computer technology such as improvement of user satisfaction, diversification of meal proposals, optimal meal selection support according to health and psychological state, and optimization of overall system computation and communication load by combining multidimensional emotion analysis and dynamic menu optimization by AI models. Specific application fields include home health management systems, patient meal proposals in remote medical care, corporate health management support, performance meal management for athletes, and mental health support systems. Furthermore, by changing the architecture of the emotion estimation AI model and the proposal algorithm (e.g., user response optimization by reinforcement learning, linkage with ingredient databases), flexible system expansion according to user groups and usage environments is possible.

The provision unit can estimate the user's emotion and adjust the instructions to the restaurant based on the estimated emotion of the user. For example, if the user is relaxed, the provision unit instructs the restaurant to use the normal cooking time. Additionally, if the user is in a hurry, the provision unit can instruct the restaurant to cook quickly. Furthermore, if the user is feeling stressed, the provision unit can instruct the restaurant to provide special services. By adjusting the instructions to the restaurant based on the user's emotion, the provision unit can provide services suitable for the user's situation. Specifically, the provision unit accepts, as input data from the user, voice data (e.g., speech audio spectrum tensor, 3 seconds in length, 16 kHz sampling rate), facial expression images (e.g., face image 128×128 pixels RGB tensor), and text chat history (e.g., natural language sentences such as “I'm in a hurry today”, “I want to relax”). The provision unit preprocesses these multimodal data with a preprocessing module for noise removal and normalization, extracts audio features (e.g., MFCC vectorization), image features (e.g., facial landmark detection), and text embeddings (e.g., BERT-based contextual vectorization). The provision unit concatenates these features into a multidimensional tensor (e.g., audio 128 dimensions+image 256 dimensions+text 768 dimensions=total 1152 dimensions) and inputs it to an emotion estimation AI model. As the AI model, a multimodal Transformer or a combination of convolutional neural networks and recurrent neural networks is used to output emotion categories (e.g., stress, relaxation, hurry, etc.) and emotion intensity scores (e.g., hurry level 0.90, relaxation level 0.80, etc.) from the input data. Examples of output include “Emotion label: hurry, intensity 0.85” and “Emotion label: relaxation, intensity 0.92”. The provision unit inputs these AI outputs to a threshold judgment module, and if the hurry level is high, adds flags such as “cooking priority: high” and “cooking time reduction instruction” to the restaurant; if the relaxation level is high, adds instructions such as “normal cooking” and “relaxed service”; and if the stress level is high, automatically generates instructions for special services (e.g., additional wet towel, message card attachment, etc.). Furthermore, the provision unit can continuously improve the accuracy of instruction optimization by learning from the user's past emotion transition history and restaurant service satisfaction scores. As a technical effect, the provision unit, unlike simple uniform instructions or manual adjustment by humans, achieves essential improvements in computer technology such as improvement of service quality, maximization of user satisfaction, optimization of restaurant operations, and optimization of overall system computation and communication load through multidimensional emotion analysis and dynamic instruction optimization by AI models. Specific application fields include home health management systems, corporate health management support, performance meal management for athletes, patient meal supply in remote medical care, and mental health support systems. Furthermore, by changing the architecture of the emotion estimation AI model and the instruction optimization algorithm (e.g., user response optimization by reinforcement learning), flexible system expansion according to user groups and usage environments is possible.

The delivery unit can estimate the user's emotion and adjust the timing of delivery based on the estimated emotion of the user. For example, if the user is in a hurry, the delivery unit shortens the delivery time. Additionally, if the user is relaxed, the delivery unit can set the normal delivery time. Furthermore, if the user is feeling stressed, the delivery unit can flexibly adjust the delivery time. By adjusting the timing of delivery based on the user's emotion, the delivery unit can provide delivery services suitable for the user's situation. Specifically, the delivery unit accepts, as input data from the user, voice data (e.g., speech audio spectrum tensor, 3 seconds in length, 16 kHz sampling rate), facial expression images (e.g., face image 128×128 pixels RGB tensor), and text chat history (e.g., natural language sentences such as “I'm in a hurry today”, “High stress”). The delivery unit preprocesses these multimodal data with a preprocessing module for noise removal and normalization, extracts audio features (e.g., MFCC vectorization), image features (e.g., facial landmark detection), and text embeddings (e.g., BERT-based contextual vectorization). The delivery unit concatenates these features into a multidimensional tensor (e.g., audio 128 dimensions+image 256 dimensions+text 768 dimensions=total 1152 dimensions) and inputs it to an emotion estimation AI model. As the AI model, a multimodal Transformer or a combination of convolutional neural networks and recurrent neural networks is used to output emotion categories (e.g., stress, relaxation, hurry, etc.) and emotion intensity scores (e.g., hurry level 0.90, stress level 0.80, etc.) from the input data. Examples of output include “Emotion label: hurry, intensity 0.85” and “Emotion label: relaxation, intensity 0.92”. The delivery unit inputs these AI outputs to a threshold judgment module, and if the hurry level is high, issues instructions to the delivery scheduler such as “select shortest delivery route” and “assign priority delivery slot”; if the relaxation level is high, issues instructions such as “standard delivery slot” and “normal route”; and if the stress level is high, automatically applies special measures such as “flexible delivery time” and “advance contact by delivery person”. Furthermore, the delivery unit can continuously improve the accuracy of timing adjustment by learning from the user's past emotion transition history and delivery satisfaction scores. As a technical effect, the delivery unit, unlike simple uniform delivery or manual adjustment by humans, achieves essential improvements in computer technology such as improvement of delivery efficiency, maximization of user satisfaction, reduction of delivery errors, and optimization of overall system computation and communication load through multidimensional emotion analysis and dynamic timing optimization by AI models. Specific application fields include home health management systems, corporate welfare services, performance meal delivery for athletes, patient meal supply in remote medical care, and mental health support systems. Furthermore, by changing the architecture of the emotion estimation AI model and the timing optimization algorithm (e.g., user response optimization by reinforcement learning), flexible system expansion according to user groups and usage environments is possible.

The acquisition unit can estimate the user's emotion and adjust the method of acquiring health information based on the estimated emotion of the user. For example, if the user is relaxed, the acquisition unit acquires detailed health information. Additionally, if the user is in a hurry, the acquisition unit can acquire concise health information. Furthermore, if the user is feeling stressed, the acquisition unit can temporarily suspend the acquisition of health information. By adjusting the method of acquiring health information based on the user's emotion, the acquisition unit can acquire health information suitable for the user's situation. Specifically, the acquisition unit accepts, as input data from the user, voice data (e.g., speech audio spectrum tensor, 3 seconds in length, 16 kHz sampling rate), facial expression images (e.g., face image 128×128 pixels RGB tensor), and text chat history (e.g., natural language sentences such as “I'm relaxed today”, “I'm in a hurry”). The acquisition unit preprocesses these multimodal data with a preprocessing module for noise removal and normalization, extracts audio features (e.g., MFCC vectorization), image features (e.g., facial landmark detection), and text embeddings (e.g., BERT-based contextual vectorization). The acquisition unit concatenates these features into a multidimensional tensor (e.g., audio 128 dimensions+image 256 dimensions+text 768 dimensions=total 1152 dimensions) and inputs it to an emotion estimation AI model. As the AI model, a multimodal Transformer or a combination of convolutional neural networks and recurrent neural networks is used to output emotion categories (e.g., stress, relaxation, hurry, etc.) and emotion intensity scores (e.g., relaxation level 0.90, hurry level 0.80, etc.) from the input data. Examples of output include “Emotion label: relaxation, intensity 0.92” and “Emotion label: hurry, intensity 0.85”. The acquisition unit inputs these AI outputs to a threshold judgment module, and if the relaxation level is high, details the health information acquisition request (e.g., acquisition of all items such as weight, blood pressure, blood glucose, sleep data, etc.); if the hurry level is high, acquires only major items (e.g., blood pressure, blood glucose, etc.); and if the stress level is high, temporarily stops or reduces the frequency of acquisition requests. Furthermore, the acquisition unit can continuously improve the accuracy of acquisition method optimization by learning from the user's past emotion transition history and health information acquisition history. As a technical effect, the acquisition unit, unlike simple uniform acquisition or manual adjustment by humans, achieves essential improvements in computer technology such as reduction of user burden, prevention of missing important information, optimization of overall system computation and communication load, and improvement of the usefulness of acquired data through multidimensional emotion analysis and dynamic acquisition method optimization by AI models. Specific application fields include home health management systems, patient monitoring in remote medical care, corporate health management support, performance management for athletes, and mental health support systems. Furthermore, by changing the architecture of the emotion estimation AI model and the acquisition method optimization algorithm (e.g., user response optimization by reinforcement learning), flexible system expansion according to user groups and usage environments is possible.

The analysis unit can estimate the user's emotion and adjust the notification method of analysis results based on the estimated emotion of the user. For example, if the user is relaxed, the analysis unit notifies detailed analysis results. Additionally, if the user is in a hurry, the analysis unit can notify concise analysis results. Furthermore, if the user is feeling stressed, the analysis unit can notify visually easy-to-understand analysis results. By adjusting the notification method of analysis results based on the user's emotion, the analysis unit can provide analysis results suitable for the user's situation. Specifically, the analysis unit accepts, as input data from the user, voice data (e.g., speech audio spectrum tensor, 3 seconds in length, 16 kHz sampling rate), facial expression images (e.g., face image 128×128 pixels RGB tensor), and text chat history (e.g., natural language sentences such as “I'm relaxed today”, “I'm in a hurry”). The analysis unit preprocesses these multimodal data with a preprocessing module for noise removal and normalization, extracts audio features (e.g., MFCC vectorization), image features (e.g., facial landmark detection), and text embeddings (e.g., BERT-based contextual vectorization). The analysis unit concatenates these features into a multidimensional tensor (e.g., audio 128 dimensions+image 256 dimensions+text 768 dimensions=total 1152 dimensions) and inputs it to an emotion estimation AI model. As the AI model, a multimodal Transformer or a combination of convolutional neural networks and recurrent neural networks is used to output emotion categories (e.g., stress, relaxation, hurry, etc.) and emotion intensity scores (e.g., relaxation level 0.90, hurry level 0.80, etc.) from the input data. Examples of output include “Emotion label: relaxation, intensity 0.92” and “Emotion label: hurry, intensity 0.85”. The analysis unit inputs these AI outputs to a threshold judgment module, and if the relaxation level is high, notifies analysis results with detailed numerical values and explanatory text; if the hurry level is high, notifies only the main points in bullet form; and if the stress level is high, notifies visually easy-to-understand analysis results using graphs and icons. Furthermore, the analysis unit can continuously improve the accuracy of notification method optimization by learning from the user's past emotion transition history and analysis result viewing history. As a technical effect, the analysis unit, unlike simple template switching or uniform notification by humans, achieves essential improvements in computer technology such as reduction of user burden, improvement of understanding of analysis results, maximization of information transmission efficiency, and optimization of overall system computation and communication load through multidimensional emotion analysis and dynamic notification method optimization by AI models. Specific application fields include home health management systems, patient analysis reports in remote medical care, corporate health management support, performance analysis for athletes, and mental health support systems. Furthermore, by changing the architecture of the emotion estimation AI model and the notification method optimization algorithm (e.g., user response optimization by reinforcement learning), flexible system expansion according to user groups and usage environments is possible.

The acquisition unit can analyze the user's past meal history and reflect it in the proposal of meal contents. For example, the acquisition unit preferentially proposes menus that the user has liked in the past. Additionally, the acquisition unit can exclude menus that the user has avoided in the past from proposals. Furthermore, the acquisition unit can adjust the proposal contents by considering nutritional balance from the user's past meal history. By analyzing the user's past meal history, the acquisition unit can propose more appropriate meal contents. Specifically, the acquisition unit collects meal history data for each user over the past year (e.g., menu ID, intake date and time, nutrient vector, satisfaction score, allergy reaction history, etc. as a structured table). The acquisition unit inputs these history data as time series vectors or category label arrays (e.g., 20 Japanese meals, 15 Western meals, 10 salads, etc.) to the AI model. As the AI model, a recurrent neural network for time series analysis or a decision tree-based gradient boosting model is used to output preference scores for each menu (e.g., grilled fish set meal 0.95, curry rice 0.80, etc.) and nutritional balance evaluation values (e.g., excessive protein intake, vitamin deficiency, etc.) from the input data. Examples of input include “Past 30 days: grilled fish set meal 5 times, curry rice 3 times, salad 2 times”. The AI model generates outputs such as “Recommended menu: grilled fish set meal, salad; excluded menu: curry rice” from these inputs. The acquisition unit automatically generates the optimal menu proposal list for each user based on the AI output and dynamically changes the system settings. Furthermore, when the acquisition unit detects changes in the user's lifestyle (e.g., introduction of new dietary restrictions, change in health goals), it can perform relearning or reevaluation and continuously optimize the proposal contents. As a technical effect, the acquisition unit, unlike human heuristics or uniform proposals, achieves essential improvements in computer technology such as improvement of proposal accuracy, reduction of user burden, maximization of preference reflection, and reduction of system operation costs through history data analysis and dynamic optimization by AI models. Specific application fields include home health management systems, corporate health management support, patient meal proposals in medical institutions, performance meal management for athletes, and proposal optimization in remote medical care. Furthermore, by changing the algorithms and feature design of the AI model (e.g., addition of user attributes, meal history, health goals) and the proposal logic (e.g., dynamic optimization by multi-armed bandit), flexible system expansion according to user groups and usage environments is possible.

The provision unit can select the optimal restaurant by considering the user's geographic location information. For example, the provision unit preferentially selects restaurants close to the user's home. Additionally, the provision unit can select restaurants that can shorten delivery time based on the user's geographic location information. Furthermore, the provision unit can select restaurants that provide local specialties by considering the user's geographic location information. By considering the user's geographic location information, the provision unit can select more appropriate restaurants. Specifically, the provision unit accepts, as input data from the user, geographic location information (e.g., GPS coordinate vector, address string, region ID), environmental information of the current location (e.g., traffic congestion level, weather, road conditions), and a list of candidate restaurants (e.g., store ID, location, provided menu, delivery range, specialty flag, etc.). The provision unit normalizes and standardizes this information with a preprocessing module and generates geographic feature vectors (e.g., latitude, longitude, elevation, region cluster ID, etc.). The provision unit combines these with the list of candidate restaurants and inputs them to the AI model. As the AI model, a large language model with a geographic information embedding layer or a graph neural network is used to output “restaurant selection score”, “predicted delivery time”, “specialty provision flag”, etc. from the input data. Examples of input include “Current location: Shinjuku, Tokyo; high traffic congestion; specialty: Shinjuku-grown vegetables”. The AI model generates outputs such as “Recommended restaurant: Store A (delivery time 15 minutes, specialty available), Store B (delivery time 20 minutes)” from these inputs, and the provision unit automatically selects the optimal restaurant according to geographic conditions based on the output. Furthermore, the provision unit can continuously improve the accuracy of selection by learning from the user's past geographic movement history and restaurant usage history (e.g., usage frequency, satisfaction score). As a technical effect, the provision unit, unlike simple item selection or uniform restaurant selection by humans, achieves essential improvements in computer technology such as maximization of delivery efficiency, minimization of user burden, optimization of overall system computation and communication load, and improvement of food service utility through multidimensional geographic information analysis and dynamic restaurant selection optimization by AI models. Specific application fields include home health management, corporate health management support, performance meal delivery for athletes, patient meal supply in remote medical care, and region-specific restaurant selection. Furthermore, by changing the algorithms and feature design of the AI model (e.g., addition of geographic and environmental factors) and the restaurant selection logic (e.g., optimization by reinforcement learning), flexible system expansion according to user groups and usage environments is possible.

The delivery unit can analyze the user's past delivery history and adjust the delivery frequency. For example, if the user frequently uses the service, the delivery unit proposes a regular delivery schedule. Additionally, if the user wishes delivery on specific days of the week or at specific times, the delivery unit can set a delivery schedule according to those preferences. Furthermore, the delivery unit can propose deliveries tailored to specific events or seasons based on the user's past delivery history. By analyzing the user's past delivery history, the delivery unit can provide a more appropriate delivery schedule. Specifically, the delivery unit collects delivery history data for each user over the past year (e.g., delivery means ID, delivery date and time, delivery duration, delivery success rate, user satisfaction score, delivery person ID, delivery cost, etc. as a structured table). The delivery unit inputs these history data as time series vectors or category label arrays (e.g., 10 Monday deliveries, 5 Friday deliveries, 3 event deliveries, etc.) to the AI model. As the AI model, a recurrent neural network for time series analysis or a decision tree-based gradient boosting model is used to output delivery frequency scores (e.g., twice a week, four times a month, etc.), optimal delivery timing (e.g., Monday morning, Friday night, etc.), and event linkage flags (e.g., birthday, holiday, etc.) from the input data. Examples of input include “Past 30 days: 3 Monday deliveries, 2 Friday deliveries, 1 birthday delivery”. The AI model generates outputs such as “Recommended delivery frequency: twice a week; recommended days: Monday and Friday; event delivery: birthday” from these inputs. The delivery unit automatically generates the optimal delivery schedule for each user based on the AI output and dynamically changes the system settings. Furthermore, when the delivery unit detects changes in the user's lifestyle (e.g., introduction of new delivery means, change in daily rhythm), it can perform relearning or reevaluation and continuously optimize the delivery frequency. As a technical effect, the delivery unit, unlike human heuristics or uniform settings, achieves essential improvements in computer technology such as improvement of delivery accuracy, reduction of user burden, minimization of delivery failure rate, and reduction of system operation costs through history data analysis and dynamic optimization by AI models. Specific application fields include home health management systems, corporate welfare services, patient meal delivery in medical institutions, performance meal management for athletes, and delivery optimization in remote medical care. Furthermore, by changing the algorithms and feature design of the AI model (e.g., addition of user attributes, delivery means performance, environmental factors) and the delivery frequency optimization logic (e.g., optimization by reinforcement learning), flexible system expansion according to user groups and usage environments is possible.

The analysis unit can adjust the nutritional balance of meal contents based on the user's health information. For example, the analysis unit may propose meals containing specific nutrients in greater amounts according to the user's health condition. Additionally, the intake of calories and salt can be adjusted in accordance with the user's health goals. Furthermore, the analysis unit can propose menus that accommodate allergies and dietary restrictions based on the user's health information. Thus, the analysis unit is able to provide meal contents with a more appropriate nutritional balance based on the user's health information. Specifically, the analysis unit receives input data from the user, such as health checkup results (e.g., blood test value vectors, BMI values, blood pressure, blood glucose levels, etc.), health goals (e.g., weight loss, muscle strengthening, blood pressure management, etc.), allergy information (e.g., egg allergy, dairy restriction, etc.), and dietary restriction information (e.g., low carbohydrate, low salt, etc.). The analysis unit normalizes and categorizes this information to generate nutrient intake vectors and restriction flags. The analysis unit inputs the health information and a list of meal candidates (e.g., dish names, nutrient amounts, allergy risks, etc.) into an AI model. The AI model may be a large language model with a health information weighting layer or a multimodal encoder with an attention mechanism, and outputs such as “nutritional balance optimization score,” “proposed menu list,” and “allergy caution flag” are generated from the input data. Examples of input include “high blood glucose, goal: weight loss, allergy: egg.” The AI model generates outputs such as “recommended menu: low-carb salad, chicken dish; excluded menu: egg dishes,” and the analysis unit automatically generates meal contents optimal for the user's health condition and goals based on these outputs. Furthermore, the analysis unit can continuously improve the accuracy of nutritional balance adjustment by learning from the user's past health information, meal history, and feedback from medical professionals. As a technical effect, unlike simple uniform proposals or manual adjustments by humans, the analysis unit achieves essential improvements in computer technology, such as reduction of health risks, support for goal achievement, reduction of user burden, and optimization of overall system computation and communication load, through multidimensional health information analysis and dynamic nutritional balance optimization by AI models. Specific application fields include home health management systems, patient meal proposals in medical institutions, corporate health management support, performance meal management for athletes, and personalized nutrition guidance. Furthermore, by changing the AI model's algorithm or health information weighting design (e.g., linkage with medical guidelines, association with user attributes), flexible system expansion according to user groups and usage environments is possible.

The provision unit can analyze the user's social media activity and reflect it in the proposal of meal contents. For example, the provision unit may propose preferred menus based on photos and comments about meals shared by the user on social media. Additionally, menus reflecting restaurants and culinary trends followed by the user can be proposed. Furthermore, the provision unit can propose meal contents tailored to specific events or seasons based on the user's social media activity. Thus, by analyzing the user's social media activity, the provision unit can propose more appropriate meal contents. Specifically, the provision unit receives input data from the user, such as social media post data (e.g., text posts, image posts, video posts, posting time, hashtags, number of likes, number of comments, and other structured data), follow relationships and trend information (e.g., followed restaurant IDs, health-related influencer IDs, trending health topic labels). The provision unit preprocesses this data using natural language processing modules and image analysis modules to generate text embedding vectors (e.g., BERT-based context vectors), image feature vectors (e.g., ResNet-based image embeddings), and post metadata vectors (e.g., posting frequency, time distribution, etc.). The provision unit concatenates these features into a multidimensional tensor (e.g., text 768 dimensions+image 256 dimensions+metadata 32 dimensions=total 1056 dimensions) and inputs it into an AI model. The AI model may be a multimodal Transformer or a graph neural network, and outputs such as meal preference estimation, trend reflection score, and event linkage flag are generated from the input data. Examples of input include “recent posts: ‘I like salad’, ‘on a diet’; image: salad photo; hashtag: #health-conscious.” The AI model generates outputs such as “recommended menu: salad, low-calorie dishes; event proposal: summer limited menu.” Based on the AI output, the provision unit suppresses unnecessary menu proposals and preferentially proposes meal contents related to the user's interests and health condition. Furthermore, the provision unit can continuously improve proposal accuracy and trend reflection accuracy by learning from the user's past social media activity history and proposal results (e.g., proposal success rate, user satisfaction score). As a technical effect, unlike simple item selection or uniform proposals by humans, the provision unit achieves essential improvements in computer technology, such as reduction of data noise, minimization of user burden, optimization of overall system computation and communication load, and enhancement of the usefulness of proposed contents, through multidimensional social data analysis and dynamic proposal optimization by AI models. Specific application fields include home health management, corporate health management support, personalized nutrition guidance, proposal optimization in telemedicine, and health trend analysis. Furthermore, by changing the AI model's algorithm or feature design (e.g., addition of post content, images, trend factors), and proposal logic (e.g., optimization by reinforcement learning), flexible system expansion according to user groups and usage environments is possible.

Below, the processing flow of Example of the Embodiment is briefly described. Specifically, the present system operates in cooperation among the acquisition unit, analysis unit, provision unit, and delivery unit, and realizes personalized meal proposals, health management, and delivery services by multidimensionally analyzing and utilizing diverse data such as the user's health information, emotion, geographic information, and social media activity. The acquisition unit collects multimodal data such as body weight, blood pressure, meal contents, amount of exercise, voice, image, and text from wearable devices and smartphone applications, and performs noise removal, normalization, and feature extraction in a preprocessing module. The analysis unit inputs the multidimensional feature tensor received from the acquisition unit into a multimodal AI model (e.g., Transformer, graph neural network, etc.) and performs emotion estimation, health status analysis, nutritional balance evaluation, and meal proposal generation. The provision unit dynamically optimizes instructions to restaurants and proposals to users based on the output results of the analysis unit, and performs restaurant selection, menu generation, and trend reflection considering geographic information and social media activity. The delivery unit automatically selects the optimal delivery means, timing, and route using an AI model based on instructions from the provision unit and the user's emotion, geographic information, and past history, and executes the delivery service. Each module is linked via an API-based interface, and the learning method and feature design of the AI model can be flexibly changed. As a technical effect, unlike conventional health management systems with single-purpose and fixed configurations, the present system can flexibly apply various combinations of AI models, data flows, interfaces, and algorithms, thereby greatly improving system scalability, maintainability, and adaptability, maximizing personalization for each user, reducing operational costs, and enabling immediate response to the introduction of new technologies in the future, thus achieving essential improvements in computer technology. Specific application fields include home health management, corporate health management support, patient monitoring in medical institutions, performance management for athletes, telemedicine, health and meal supply support during disasters, region-specific health risk management, and personalized nutrition guidance.

Step 1: The acquisition unit acquires health information of the user. The user's health information includes body weight, blood pressure, meal contents, and amount of exercise. The acquisition unit collects health information entered by the user and data obtained from wearable devices, and provides it to the analysis unit. Step 2: The analysis unit analyzes the user's preferences and nutritional balance based on the information acquired by the acquisition unit. The analysis unit generates optimal meal contents using generative AI based on the user's past meal history and health status. Step 3: The provision unit provides the meal contents proposed by the analysis unit to a restaurant. The provision unit automatically transmits the proposed meal contents to the restaurant and generates menus using generative AI. Step 4: The delivery unit delivers the meal provided by the provision unit to the user. The delivery unit delivers the meal prepared by the restaurant to the user's home and calculates the optimal delivery route using generative AI. Specifically, the acquisition unit receives input data from the user, such as numerical vectors and categorical data for body weight, blood pressure, meal contents, and amount of exercise, time-series data from wearable devices (e.g., daily step count arrays, time-series sleep scores, etc.), and multimodal data such as voice, image, and text. The acquisition unit performs noise removal, normalization, and feature extraction in a preprocessing module for these data and generates multidimensional tensors (e.g., body weight 1 dimension+blood pressure 2 dimensions+meal contents vector+amount of exercise 1 dimension+voice 128 dimensions+image 256 dimensions+text 768 dimensions, etc.). The analysis unit inputs these feature tensors into a multimodal AI model (e.g., Transformer, graph neural network, etc.) and performs emotion estimation (e.g., stress level 0.85, relaxation level 0.10, etc.), health status analysis (e.g., blood pressure anomaly detection, nutritional balance evaluation), and meal proposal generation (e.g., recommended menu list, allergy caution flag, etc.). The provision unit dynamically optimizes instructions to restaurants (e.g., cooking priority, special service instructions, etc.) and proposals to users (e.g., detailed menu, summary proposal, etc.) based on the output results of the analysis unit, and performs restaurant selection, menu generation, and trend reflection considering geographic information and social media activity. The delivery unit automatically selects the optimal delivery means, timing, and route using an AI model based on instructions from the provision unit and the user's emotion, geographic information, and past history, and executes the delivery service. Each module is linked via an API-based interface, and the learning method and feature design of the AI model can be flexibly changed. As a technical effect, unlike conventional health management systems with single-purpose and fixed configurations, the present system can flexibly apply various combinations of AI models, data flows, interfaces, and algorithms, thereby greatly improving system scalability, maintainability, and adaptability, maximizing personalization for each user, reducing operational costs, and enabling immediate response to the introduction of new technologies in the future, thus achieving essential improvements in computer technology. Specific application fields include home health management, corporate health management support, patient monitoring in medical institutions, performance management for athletes, telemedicine, health and meal supply support during disasters, region-specific health risk management, and personalized nutrition guidance.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned acquisition unit, analysis unit, provision unit, and delivery unit is implemented by at least one of, for example, a smart device 14 and a data processing apparatus 12. For example, the acquisition unit is implemented by a control unit 46A of the smart device 14 and collects health information input by the user and data from a wearable device. The analysis unit is implemented, for example, by a specific processing unit 290 of the data processing apparatus 12 and analyzes the user's preferences and nutritional balance based on the collected information. The provision unit is implemented, for example, by the specific processing unit 290 of the data processing apparatus 12 and provides the meal contents proposed based on the analysis results to a restaurant. The delivery unit is implemented, for example, by the control unit 46A of the smart device 14 and delivers the meal prepared by the restaurant to the user's home. The correspondence between each unit and the apparatus or control unit is not limited to the examples described above and various modifications are possible.

### Second Embodiment

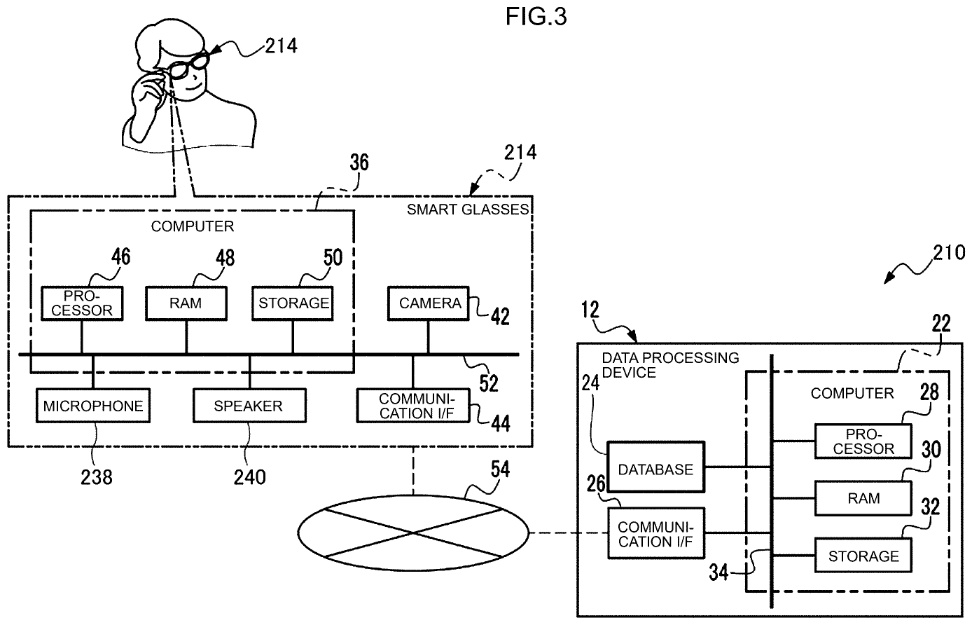

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

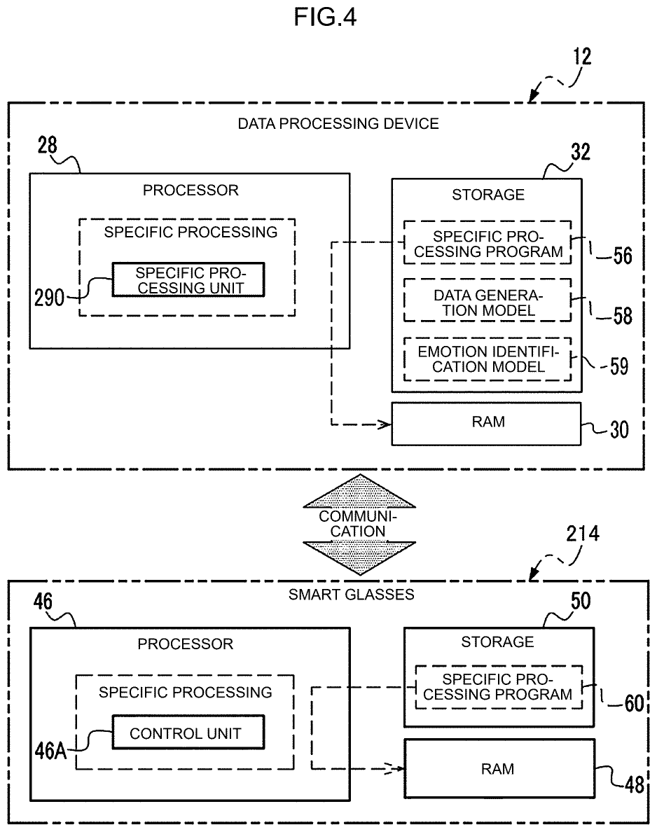

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned acquisition unit, analysis unit, provision unit, and delivery unit is implemented by at least one of, for example, smart glasses 214 and a data processing apparatus 12. For example, the acquisition unit is implemented by a control unit 46A of the smart glasses 214 and collects health information input by the user and data from a wearable device. The analysis unit is implemented, for example, by a specific processing unit 290 of the data processing apparatus 12 and analyzes the user's preferences and nutritional balance based on the collected information. The provision unit is implemented, for example, by the specific processing unit 290 of the data processing apparatus 12 and provides the meal contents proposed based on the analysis results to a restaurant. The delivery unit is implemented, for example, by the control unit 46A of the smart glasses 214 and delivers the meal prepared by the restaurant to the user's home. The correspondence between each unit and the apparatus or control unit is not limited to the examples described above and various modifications are possible.

### Third Embodiment

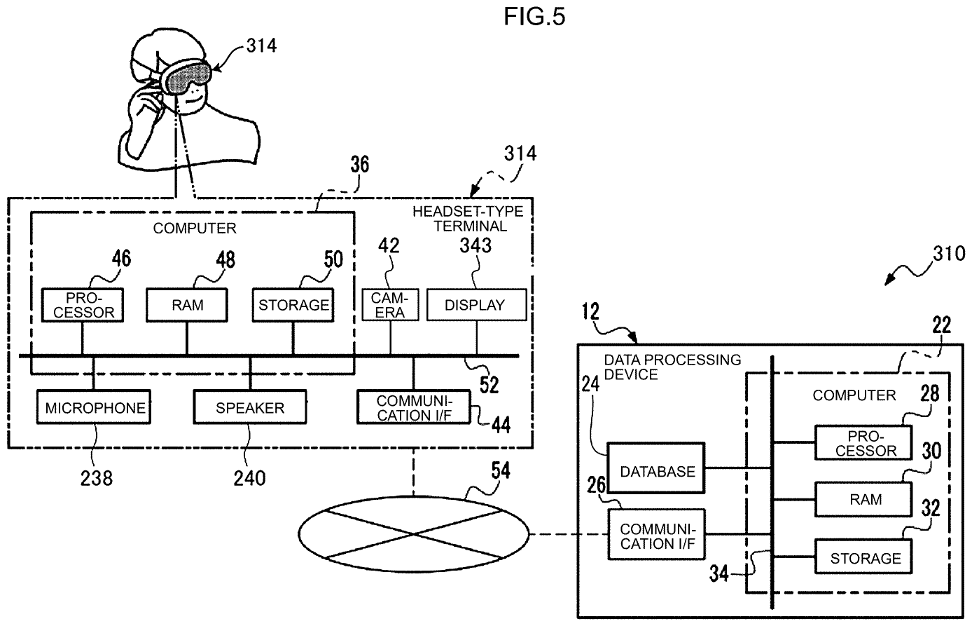

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

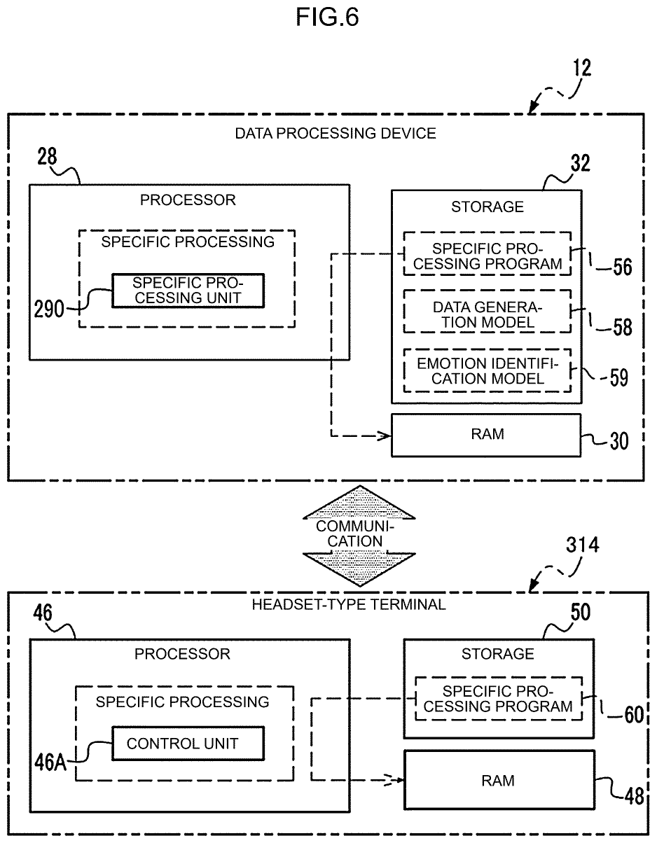

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned acquisition unit, analysis unit, provision unit, and delivery unit is implemented by at least one of, for example, a headset-type terminal 314 and a data processing apparatus 12. For example, the acquisition unit is implemented by a control unit 46A of the headset-type terminal 314 and collects health information input by the user and data from a wearable device. The analysis unit is implemented, for example, by a specific processing unit 290 of the data processing apparatus 12 and analyzes the user's preferences and nutritional balance based on the collected information. The provision unit is implemented, for example, by the specific processing unit 290 of the data processing apparatus 12 and provides the meal contents proposed based on the analysis results to a restaurant. The delivery unit is implemented, for example, by the control unit 46A of the headset-type terminal 314 and delivers the meal prepared by the restaurant to the user's home. The correspondence between each unit and the apparatus or control unit is not limited to the examples described above and various modifications are possible.

### Fourth Embodiment

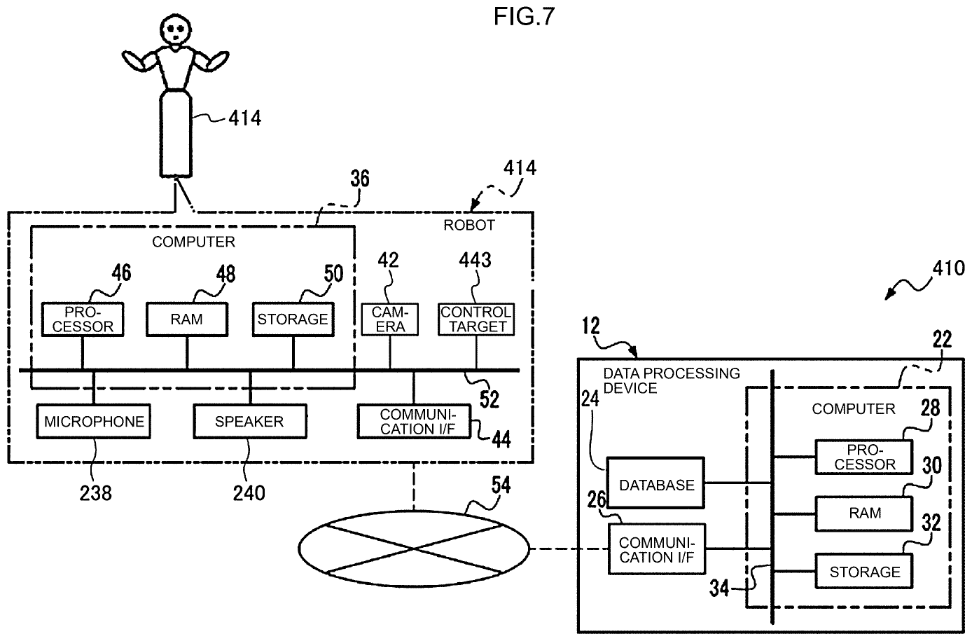

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

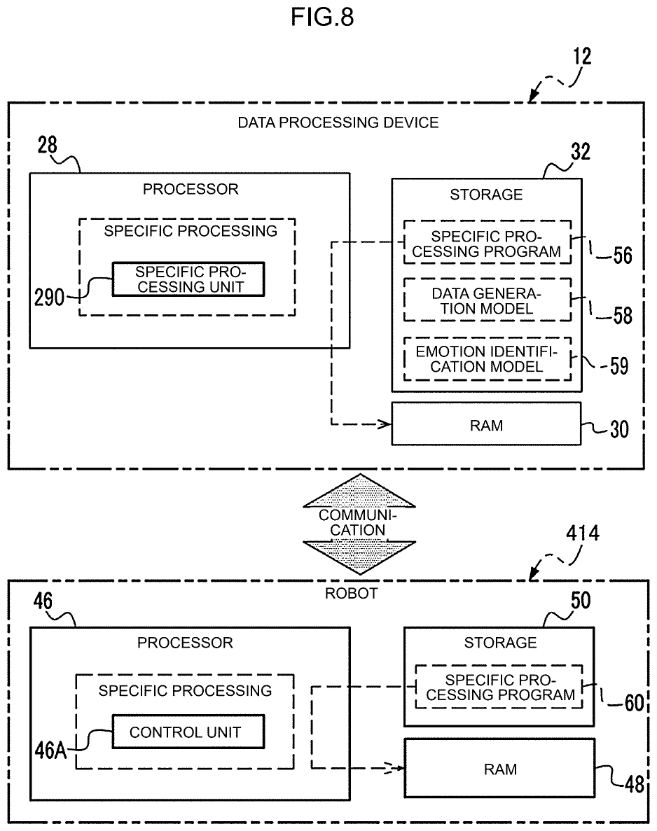

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned acquisition unit, analysis unit, provision unit, and delivery unit is implemented by at least one of, for example, a robot 414 and a data processing apparatus 12. For example, the acquisition unit is implemented by a control unit 46A of the robot 414 and collects health information input by the user and data from a wearable device. The analysis unit is implemented, for example, by a specific processing unit 290 of the data processing apparatus 12 and analyzes the user's preferences and nutritional balance based on the collected information. The provision unit is implemented, for example, by the specific processing unit 290 of the data processing apparatus 12 and provides the meal contents proposed based on the analysis results to a restaurant. The delivery unit is implemented, for example, by the control unit 46A of the robot 414 and delivers the meal prepared by the restaurant to the user's home. The correspondence between each unit and the apparatus or control unit is not limited to the examples described above and various modifications are possible.

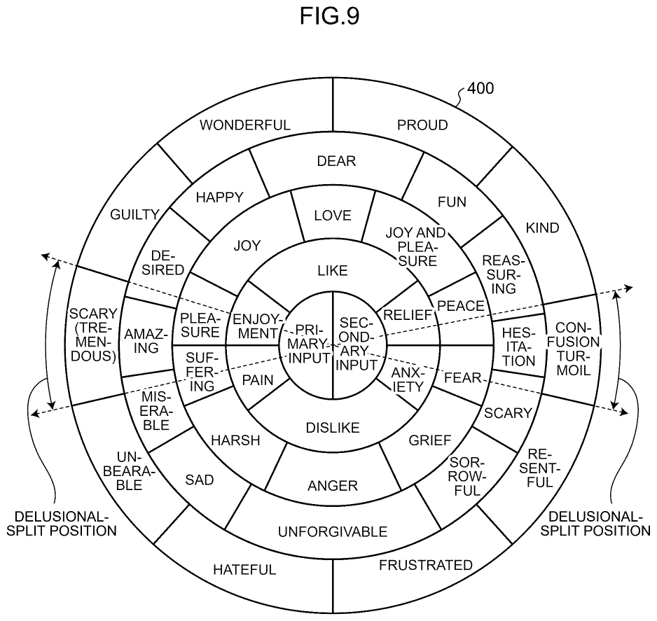

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

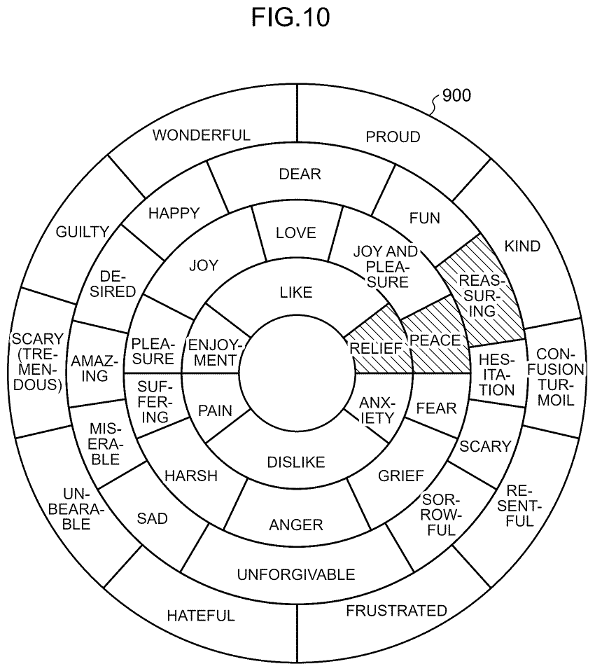

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

(Supplementary Note 1) A system comprising: an acquisition unit configured to acquire health information of a user; an analysis unit configured to analyze the user's preferences and nutritional balance based on the information acquired by the acquisition unit; a provision unit configured to provide meal contents proposed by the analysis unit to a restaurant; and a delivery unit configured to deliver the meal provided by the provision unit to the user.

(Supplementary Note 2) The acquisition unit is configured to acquire the user's preferences, allergy information, and health status.

(Supplementary Note 3) The analysis unit is configured to generate appropriate meal contents based on the user's past meal history and health status.

(Supplementary Note 4) The provision unit is configured to automatically transmit the proposed meal contents to the restaurant; and the system according to Supplementary Note 1.

(Supplementary Note 5) The delivery unit is configured to deliver the meal prepared by the restaurant to the user's home; and the system according to Supplementary Note 1.

(Supplementary Note 6) The acquisition unit is configured to estimate the user's emotion and adjust the timing of acquiring health information based on the estimated emotion of the user; and the system according to Supplementary Note 1.

(Supplementary Note 7) The acquisition unit is configured to analyze the user's past health information and select an appropriate acquisition method.

(Supplementary Note 8) The acquisition unit is configured to perform filtering based on the user's current lifestyle and meal preferences when acquiring health information; and the system according to Supplementary Note 1.

(Supplementary Note 9) The acquisition unit is configured to estimate the user's emotion and determine the priority of health information to be acquired based on the estimated emotion of the user; and the system according to Supplementary Note 1.

(Supplementary Note 10) The acquisition unit is configured to preferentially acquire highly relevant information by considering the user's geographic location information when acquiring health information; and the system according to Supplementary Note 1.

(Supplementary Note 11) The acquisition unit is configured to analyze the user's social media activity and acquire relevant information when acquiring health information; and the system according to Supplementary Note 1.

(Supplementary Note 12) The analysis unit is configured to estimate the user's emotion and adjust the expression method of analysis based on the estimated emotion of the user; and the system according to Supplementary Note 1.

(Supplementary Note 13) The analysis unit is configured to adjust the level of detail of analysis based on the importance of health information during analysis; and the system according to Supplementary Note 1.

(Supplementary Note 14) The analysis unit is configured to apply different analysis algorithms according to the category of health information during analysis; and the system according to Supplementary Note 1.

(Supplementary Note 15) The analysis unit is configured to estimate the user's emotion and adjust the length of analysis based on the estimated emotion of the user; and the system according to Supplementary Note 1.

(Supplementary Note 16) The analysis unit is configured to determine the priority of analysis based on the timing of acquiring health information during analysis; and the system according to Supplementary Note 1.

(Supplementary Note 17) The analysis unit is configured to adjust the order of analysis based on the relevance of health information during analysis; and the system according to Supplementary Note 1.

(Supplementary Note 18) The provision unit is configured to estimate the user's emotion and adjust the expression method of provision based on the estimated emotion of the user; and the system according to Supplementary Note 1.

(Supplementary Note 19) The provision unit is configured to adjust the level of detail of provision based on the importance of meal contents during provision; and the system according to Supplementary Note 1.

(Supplementary Note 20) The provision unit is configured to apply different provision algorithms according to the category of meal contents during provision; and the system according to Supplementary Note 1.

(Supplementary Note 21) The provision unit is configured to estimate the user's emotion and adjust the length of provision based on the estimated emotion of the user; and the system according to Supplementary Note 1.

(Supplementary Note 22) The provision unit is configured to determine the priority of provision based on the timing of submission of meal contents during provision; and the system according to Supplementary Note 1.

(Supplementary Note 23) The provision unit is configured to adjust the order of provision based on the relevance of meal contents during provision; and the system according to Supplementary Note 1.

(Supplementary Note 24) The delivery unit is configured to estimate the user's emotion and adjust the delivery method based on the estimated emotion of the user; and the system according to Supplementary Note 1.

(Supplementary Note 25) The delivery unit is configured to analyze the user's past delivery history and select an optimal delivery method during delivery; and the system according to Supplementary Note 1.

(Supplementary Note 26) The delivery unit is configured to customize the means of delivery based on the user's current lifestyle during delivery; and the system according to Supplementary Note 1.

(Supplementary Note 27) The delivery unit is configured to estimate the user's emotion and determine the priority of delivery based on the estimated emotion of the user; and the system according to Supplementary Note 1.

(Supplementary Note 28) The delivery unit is configured to select an optimal delivery method by considering the user's geographic location information during delivery; and the system according to Supplementary Note 1.

(Supplementary Note 29) The delivery unit is configured to analyze the user's social media activity and propose means of delivery during delivery; and the system according to Supplementary Note 1.

What is claimed is:

1. A system comprising:
a communication interface configured to communicate with a client terminal via a packet-switched network conforming to at least one of a 5G, Wi-Fi, or Bluetooth communication standard;
a processor;
a random-access memory;
a storage storing a data generation model obtained by deep learning on a neural network, and an emotion identification model;
a database; and
circuitry configured to:
receive, from the client terminal via the communication interface, sensor data comprising at least one of voice data, text data, or image data;
estimate an emotion of the user by applying the emotion identification model to the sensor data;
generate, by inputting the sensor data and the estimated emotion into the data generation model, inference data comprising at least one of text data, voice data, or image data; and
transmit the inference data to the client terminal via the communication interface, the inference data causing the client terminal to present the inference data to the user.

2. The system according to claim 1,
wherein the sensor data further comprises time-series data received from a wearable device communicatively coupled to the client terminal, the time-series data comprising at least one of a heart rate, a step count, or a sleep score.

3. The system according to claim 1,
wherein the circuitry is further configured to preprocess the sensor data by performing noise removal and normalization in a preprocessing module and generating a multidimensional feature tensor from the preprocessed sensor data.

4. The system according to claim 3,
wherein the circuitry is further configured to input the multidimensional feature tensor into a multimodal AI model comprising a Transformer to perform at least one of emotion estimation, status analysis, or proposal generation.

5. The system according to claim 1,
wherein the sensor data comprises health information of the user including at least one of body weight, blood pressure, meal contents, or amount of exercise, and wherein the inference data comprises meal contents generated based on the health information and the estimated emotion.

6. The system according to claim 5,
wherein the circuitry is further configured to transmit, via the communication interface to a restaurant system coupled to the network, the generated meal contents.

7. The system according to claim 6,
wherein the circuitry is further configured to calculate, using the data generation model, a delivery route for delivering a meal prepared by the restaurant system to the user, and to transmit the delivery route to the client terminal via the communication interface.

8. The system according to claim 1,
wherein the circuitry is further configured to adjust a method of acquiring the sensor data based on the estimated emotion, such that when the estimated emotion indicates relaxation, the circuitry acquires detailed sensor data, and when the estimated emotion indicates urgency, the circuitry acquires concise sensor data.

9. The system according to claim 1,
wherein the circuitry is further configured to determine a priority of the sensor data to be acquired based on the estimated emotion.

10. The system according to claim 1,
wherein the circuitry is further configured to adjust a level of detail of the inference data based on an importance score associated with the sensor data, such that the circuitry generates detailed inference data for sensor data having a high importance score and concise inference data for sensor data having a low importance score.

11. The system according to claim 1,
wherein the circuitry is further configured to apply different analysis algorithms according to a category of the sensor data.

12. The system according to claim 1,
wherein the circuitry is further configured to adjust a length of the inference data based on the estimated emotion, such that when the estimated emotion indicates urgency, the circuitry generates a short inference result, and when the estimated emotion indicates relaxation, the circuitry generates a detailed inference result.

13. The system according to claim 1,
wherein the circuitry is further configured to select a restaurant based on geographic location information of the user received from the client terminal, and to transmit meal contents generated by the data generation model to the selected restaurant via the communication interface.

14. The system according to claim 1,
wherein the circuitry is further configured to analyze social media activity data of the user received from the client terminal, and to adjust the inference data based on the analyzed social media activity data.

15. The system according to claim 1,
wherein the circuitry is further configured to calculate, using a reinforcement learning algorithm or a graph neural network, an optimal delivery route based on at least one of geographic location information, traffic data, or weather information received via the communication interface.

16. The system according to claim 1,
wherein the circuitry is further configured to customize a delivery method based on a current lifestyle of the user, the current lifestyle comprising at least one of an occupation category, a working hour, or an activity status received from the client terminal.

17. The system according to claim 1,
wherein the data generation model comprises at least one of a text generation AI, an image generation AI, or a multimodal generation AI, and wherein the circuitry is further configured to output the inference data in at least one of a voice data format, a text data format, or an image data format.

18. A system comprising:
a communication interface configured to communicate, via a packet-switched network conforming to at least one of a 5G, Wi-Fi, or Bluetooth communication standard, with a client terminal comprising a microphone, a speaker, a camera having a CMOS image sensor, and a display;
a processor;
a random-access memory;
a storage storing a data generation model obtained by deep learning on a neural network, and an emotion identification model;
a database; and
circuitry configured to:
receive, from the client terminal via the communication interface, sensor data comprising at least one of voice data captured by the microphone, text data, or image data captured by the camera;
preprocess the sensor data by performing noise removal and normalization to generate a multidimensional feature tensor;
estimate an emotion of the user by applying the emotion identification model to the sensor data;
generate, by inputting the multidimensional feature tensor and the estimated emotion into the data generation model, inference data comprising at least one of text data, voice data, or image data;
transmit the inference data to the client terminal via the communication interface, the inference data causing the client terminal to present the inference data to the user via at least one of the speaker or the display; and
receive, from the client terminal via the communication interface, voice data indicating user input responsive to the presented inference data.

19. The system according to claim 18,
wherein the data generation model comprises at least one of a text generation AI, an image generation AI, or a multimodal generation AI, and
wherein the data generation model is a fine-tuned model configured to output inference results from prompts without instructions.

20. A method performed by circuitry of a data processing system comprising a processor, a random-access memory, a storage storing a data generation model obtained by deep learning on a neural network and an emotion identification model, a database, and a communication interface, the method comprising:
receiving, from a client terminal via the communication interface and a packet-switched network conforming to at least one of a 5G, Wi-Fi, or Bluetooth communication standard, sensor data comprising at least one of voice data, text data, or image data;
estimating an emotion of the user by applying the emotion identification model to the sensor data;
generating, by inputting the sensor data and the estimated emotion into the data generation model, inference data comprising at least one of text data, voice data, or image data; and
transmitting the inference data to the client terminal via the communication interface, the inference data causing the client terminal to present the inference data to the user.
